(19) United States
(12) Patent Application Publication
(10) Pub. No.: US 2004/0009574 A1
(43) Pub. Date: Jan. 15, 2004

(54) Compositions and methods for detecting streptococcus agalactiae capsular polysaccharide synthesis genes

(76) Inventors: Nanibhushan Dattagupta, San Diego, CA (US); Ketan Shah, San Diego, CA (US)

Correspondence Address: Laurie A. Axford, Morrison & Foerster LLP, Suite 500, 3811 Valley Centre Drive, San Diego, CA, 92130-2332, US

(21) Appl. No.: 10/192,280

(22) Filed: Jul. 9, 2002

(51) Int. Cl. C12N 1/20

(52) U.S. Cl. 435/252.3

(57) ABSTRACT

This invention relates generally to Group B streptococci (GBS) Streptococcus agalactiae detection. More specifically, the present invention provides for novel probes for a specific and sensitive diagnostic test of GBS. These GBS-specific probes hybridize with the capsular polysaccharide synthesis (cps) gene. Arrays comprising the probes immobilized on a support for hybridization analysis and methods for GBS detection using the probes are also provided.

### FIELD OF THE INVENTION

This invention relates generally to Group B streptococci (GBS) detection. More specifically, the present invention provides for novel probes for a specific and sensitive diagnostic test for GBS. These GBS-specific probes hybridize with the capsular polysaccharide synthesis (cps) gene. Arrays comprising the novel probes immobilized on a support for hybridization analysis and methods for GBS detection using the probes are also provided.

### BACKGROUND OF THE INVENTION

Life-threatening bacterial infections (bacteremia, pneumonia, and meningitis) by Group B streptococci (also referred to herein as “GBS” or “Streptococcus agalactiae”) are the major cause of morbidity and mortality in neonates and very young infants (Schuchat, Clin. Microbiol. Rev. 11: 497-513 (1998)). Two to three of every 1,000 live births result in GBS infection. Approximately 75% of infections in infants occur in the first few days of life (early-onset infections), while late-onset infections typically occur in infants between 1 week and 3 months of age. Newborns with early-onset GBS disease usually acquire the organism from their GBS-colonized mothers during vaginal delivery. Therefore, prenatal screening for GBS is now recommended to reduce the incidence of early-onset GBS disease (see Committee on Infectious Diseases and Committee on Fetus and Newborn, Revised guidelines for prevention of early-onset group B streptococci (GBS) infection, Pediatrics, 99: 489-496 (1997)).

Another problem associated with GBS is that the frequent use of antibiotics during the prenatal period has led to the emergence of an antibiotic-resistant strains of GBS. Furthermore, GBS infections also pose a significant danger of sepsis and menigitis in adults that are elderly, inmunocompromised, or suffering from diabetes, cirrhosis, and malignancies. Early detection of GBS is thus critical among these patients to insure prompt and cost-effective treatment to control the infection.

The virulence of the GBS is determined by the production of an exopolysaccharide capsule, a type-specific capsular polysaccharide (cps), that prevents the deposition of host complement factors and inhibits phagocytosis. As with exopolysaccharide capsules in other species, cps synthesis is controlled by a series of genes and regulatory sequences organized in an operon. Nine distinct capsular serotypes of GBS have been identified based on immune reactivity to the capsular polysaccharide. Serotypes Ia, III, and V are currently the most common isolates of GBS in the United States that are associated with early-onset neonatal disease with type III GBS being the most prevalent. The type III GBS produces its capsular polysaccharide through the cpsIII operon that contains 16 genes involved in cps synthesis over a 15.5 kb stretch of DNA (Chaffin et al., J. Bacteriol., 182: 4466-4477 (2000)).

GBS culture identification can be done by AccuProbe (Gen-Probe) and by BACTEC 9000 MB and BACTEC 460TB (Becton Dickinson) . The former is based on the amplification and detection of the 16S ribosomal RNA. Recently, molecular tests have been developed for the rapid detection of GBS (You, U.S. Pat. No. 6,004,754 and Greisen et al., U.S. Pat. No. 5,620, 847). You's method is based on tSDA (thermophilic strand displacement amplification) and detection of a unique 336 base pair region, whereas the method by Greisen et al. is a PCR (polymerase chain reaction) based amplification of a 370 base pair region of 16S ribosomal RNA. Further, Kong et al., J. Clin. Microbiol., 40: 216-26 (2002), have reported molecular serotyping as an alternative method to conventional serotyping for GBS isolates through the use of PCR and amplicon-sequencing, which is also purportedly useful for epidemiological studies. The molecular serotype identification is based on the sequence heterogeneity of the 790 base pair region located between the 3′ end of cpsE-cpsF and the 5′ end of cpsG. Although the amplification based methods are sensitive, these protocols may carry risks associated with nucleic acid contamination that gives false positive results. Nucleic acid amplification followed by (i) hybridization-based detection of the amplicon or (ii) DNA sequencing may add complexity to the protocols.

Notwithstanding the existence of the detection methods described above, there remains a need for rapid, accurate, sensitive and simple methods for the identification of GBS. The present invention relates to the development of cps sequence-specific probes that can be used in any protocol of hybridization, with or without nucleic acid amplification. These probes are highly specific and are capable of detecting GBS safely, efficiently and reliably. They were selected based on their G+C content, length, Tm values and absence of hairpin secondary structure.

### SUMMARY OF THE INVENTION

The present invention provides for molecular probes and uses thereof for a direct, specific and sensitive diagnostic test of Streptococcus agalactiae, popularly known as Group B Streptococci (GBS), a causative agent for neonatal sepsis and neonatal meningitis. These GBS-specific probes hybridize with at least one capsular polysaccharide synthesis (cps) gene sequence that is believed to be present only in GBS.

In one aspect, the present invention provides for an oligonucleotide probe for detecting GBS in a sample comprising a nucleotide sequence that hybridizes with a target nucleotide sequence, wherein the target nucleotide sequence is all or part of the cps gene, or a complementary strand thereof, and wherein the probe has a G+C content from about 30 to 70%, a Tm value from about 40-80° C., a length of at least 8 nucleotides (if DNA and/or RNA, or 6 if PNA or other nucleic acid analog with a higher affinity than DNA or RNA) and does not contain any hairpin secondary structure. Preferably, the probe hybridizes with a target nucleotide sequence of a Streptococcus agalactiae cps gene under middle or high stringency.

In another aspect, the probe may comprise a nucleotide sequence that hybridizes under low stringency with a target comprising a nucleotide sequence, or a complementary strand thereof, selected from the group consisting of:

(SEQ ID NO:2)

5′-AACCGACCTTGATGAAGAAGTGAAATCGACCATCATTCAATTAAGAC
AGTTCGAAAAATCAGTCA GAAATCCTTT-3′
(GenBank AFB028896 125-200)

(SEQ ID NO:3)

5′-TAGACAATATCAATAACATCATCCACGTCAAGAGGGATTGCGACAAT
TTCGTCACCGTTCAATTTTGAGTTGAGGATACCAGTTGCCACTGTATAAC
CATCTAATCCAATCATTAAATTAAACAGAGTTGCTCTATCACTGACAACA
ATGGATTTTGGGTGAGGAATTTGAGACATCATTTCCTCAGAAAAGTAGAA
AGAA-3′
(GenBank AFB028896 500-700)

(SEQ ID NO:4)

5′-TAATAGCATAAGAAATTATGAATATTTAGACAAAGTGATACCAGTTG
AAGAAGCACTACTAAACATGTATGACATTAAAACTTATGGTTCAATTTTT
ATTACTGCATGGGGAAAATTATTCCATAAATCTATATTCAATGATTTAGA
ATTTGCATTAAATAAGTATCATGAGGATGAATTCTTTAACTATAAAGCAT
ACTTAAAAGCTAATTCTATAACATACATAGACAAGCCTCTCTATCATTAT
CGTATACGAGTAGGTAGTATCATGAATAATAGTGATAATGTTATAATTGC
TAGAAAGAAACTTG ATGTTTTATCAGCATTAGACGAGCGAATAA-3′
(GenBank AFB028896 10,100-10,440)

(SEQ ID NO:5)

5′-TCCAGTGTCTATTTATAGGATCCAATTCAGATACTGGTTCTGATGAT
ATTGCAAAGGCTATCAATACTTATTTGATAAATCATGAAAATAGTTATTG
CTTTGCTTCTTTATCAACCCAACTTTATCATTCCTTAATTCGTCATTCAC
TAGGACTGATTGGTAATTCGTCTTCTGGTTTAATAGAAGTTCCTTCTTTG
ATGAAACCTACTCTTAATATTGGTGATCGCCAAAAAGGTAGGCTACATGG
AGAA-3′
(GenBank AFB028896 14,900-15,150)

(SEQ ID NO:6)

5′-TCTAAAATGAAAAAAAAGTTGGCATTTTTAGGCGCAGGGAGTCATGC
TGATGCGATTGCTCCGGTTATTGATCCTCTTGTGTATGATTTCGTAGGTT
TTTTTGATGATAAAGATATTACGGAGCATGATGGTTATCCTGTTCTTGGA
AAACTTTATGATGTGCTACCTTACCTTGA-3′
(GenBank AF355776 15,300-15,475)

(SEQ ID NO:7)

5′-GTATTGACACCAGATAGTATTTGTGGACGTGGTATCTTTATTGGTTT
TGGGGCTTTTATAGGTTCTAAAGTGAAGCTGTTTGATAACAACGTTGTTA
ATAC-3′
(GenBank AF355776 15,600-15,700)

(SEQ ID NO:8)

5′-CGACAATTGGAGCAGGAGCTGTTGTTGTAAAAGATATTATAGAACCT
GGAACCTATGTAGGGGTTCCAGCAAAAAAAATAAAATAAAGGATATGTTA
TGAAGCCAATTTGTATTATTCCTGCGCGATCAGGGTCAAAAGGATTACCA
GATA-3′
(GenBank AF355776 15,850-16,000)

In yet another aspect, the probe may comprise a nucleotide sequence that hybridizes under low stringency with a target comprising a nucleotide sequence, or a complementary strand thereof, selected from the group consisting of:

| | |
|---|---|
| 5′-GCCGCACCAGTTAAAAACGATAC-3′, | (SEQ ID NO:9) |
| 5′-TGTCAGCAAATTAGCCGCACCAGT-3′, | (SEQ ID NO:10) |
| 5′-GAGTTGATGTCAACAGGTCGGTTCG-3′, | (SEQ ID NO:11) |
| 5′-GTCTAATCATTCGCGCCGTCAA-3′, | (SEQ ID NO:12) |
| 5′-CCAGGAATATAGAACGTTAGGG-3′, | (SEQ ID NO:13) |
| 5′-GCCGGTAGAGCTATTACCATC-3′, | (SEQ ID NO:14) |
| 5′-CAGCATTGTCTGATAAAGGCTATGC-3′, | (SEQ ID NO:15) |
| 5′-GATATGGTGGGAGCAGATAAAGG-3′, | (SEQ ID NO:16) |
| 5′-AGTCATGCTGATGCGATTGCTC-3′, | (SEQ ID NO:17) |
| 5′-GCGATCAGGGTCAAAAGGATTACCA-3′, | (SEQ ID NO:18) |
| 5′-CGGAGCATGATGGTTATCCTG-3′, | (SEQ ID NO:19) |
| 5′-CACCAGATAGTATTTGTGGACGTGG-3′, and | (SEQ ID NO:20) |
| 5′-ACCTATGTAGGGGTTCCAGCAA-3′. | (SEQ ID NO:21) |

In still another aspect, the present invention provides for an oligonucleotide probe for detecting GBS in a sample comprising a nucleotide sequence having at least 90% identity with a sequence selected from the group consisting of:

| | |
|---|---|
| 5′-GCCGCACCAGTTAAAAACGATAC-3′, | (SEQ ID NO:9) |
| 5′-TGTCAGCAAATTAGCCGCACCAGT-3′, | (SEQ ID NO:1O) |
| 5′-GAGTTGATGTCAACAGGTCGGTTCG-3′, | (SEQ ID NO:11) |
| 5′-GTCTAATCATTCGCGCCGTCAA-3′, | (SEQ ID NO:12) |
| 5′-CCAGGAATATAGAACGTTAGGG-3′, | (SEQ ID NO:13) |
| 5′-GCCGGTAGAGCTATTACCATC-3′, | (SEQ ID NO:14) |
| 5′-CAGCATTGTCTGATAAAGGCTATGC-3′, | (SEQ ID NO:15) |
| 5′-GATATGGTGGGAGCAGATAAAGG-3′, | (SEQ ID NO:16) |
| 5′-AGTCATGCTGATGCGATTGCTC-3′, | (SEQ ID NO:17) |
| 5′-GCGATCAGGGTCAAAAGGATTACCA-3′, | (SEQ ID NO:18) |
| 5′-CGGAGCATGATGGTTATCCTG-3′, | (SEQ ID NO:19) |
| 5′-CACCAGATAGTATTTGTGGACGTGG-3′, | (SEQ ID NO:20) |
| 5′-ACCTATGTAGGGGTTCCAGCAA-3′, | (SEQ ID NO:21) |
| 3′-CGGCGTGGTCAATTTTTGCTATC-5′, | (SEQ ID NO:22) |
| 3′-ACAGTCGTTTAATCGGCGTGGTCA-5′, | (SEQ ID NO:23) |
| 3′-CTCAACTACAGTTGTCCAGCCAAGC-5′, | (SEQ ID NO:24) |
| 3′-CAGATTAGTAAGCGCGGCAGTT-5′, | (SEQ ID NO:25) |
| 3′-GGTCCTTATATCTTGCAATCCC-5′, | (SEQ ID NO:26) |
| 3′-CGGCCATCTCGATAATGGTAG-3′, | (SEQ ID NO:27) |
| 3′-GTCGTAACAGACTATTTCCGATACG-3′, | (SEQ ID NO:28) |
| 3′-CTATACCACCCTCGTCTATTTCC-5′, | (SEQ ID NO:29) |
| 3′-TCAGTACGACTACGCTAACGAG-5′, | (SEQ ID NO:30) |
| 3′-CGCTAGTCCCAGTTTTCCTAATGGT-5′, | (SEQ ID NO:31) |
| 3′-GCCTCGTACTACCAATAGGAC-3′, | (SEQ ID NO:32) |
| 3′-GTGGTCTATCATAAACACCTGCACC-3′, and | (SEQ ID NO:33) |
| 3′-TGGATACATCCCCAAGGTCGTT-3′. | (SEQ ID NO:34) |

Preferably, the oligonucleotide probe comprises a nucleotide sequence selected from the group consisting of:

| | |
|---|---|
| 5′-GCCGCACCAGTTAAAAACGATAC-3′, | (SEQ ID NO:9) |
| 5′-TGTCAGCAAATTAGCCGCACCAGT-3′, | (SEQ ID NO:10) |
| 5′-GAGTTGATGTCAACAGGTCGGTTCG-3′, | (SEQ ID NO:11) |
| 5′-GTCTAATCATTCGCGCCGTCAA-3′, | (SEQ ID NO:12) |
| 5′-CCAGGAATATAGAACGTTAGGG-3′, | (SEQ ID NO:13) |
| 5′-GCCGGTAGAGCTATTACCATC-3′, | (SEQ ID NO:14) |
| 5′-CAGCATTGTCTGATAAAGGCTATGC-3′, | (SEQ ID NO:15) |
| 5′-GATATGGTGGGAGCAGATAAAGG-3′, | (SEQ ID NO:16) |
| 5′-AGTCATGCTGATGCGATTGCTC-3′, | (SEQ ID NO:17) |
| 5′-GCGATCAGGGTCAAAAGGATTACCA-3′, | (SEQ ID NO:18) |
| 5′-CGGAGCATGATGGTTATCCTG-3′, | (SEQ ID NO:19) |
| 5′-CACCAGATAGTATTTGTGGACGTGG-3′, | (SEQ ID NO:20) |
| 5′-ACCTATGTAGGGGTTCCAGCAA-3′, | (SEQ ID NO:21) |
| 3′-CGGCGTGGTCAATTTTTGCTATC-5′, | (SEQ ID NO:22) |
| 3′-ACAGTCGTTTAATCGGCGTGGTCA-5′, | (SEQ ID NO:23) |
| 3′-CTCAACTACAGTTGTCCAGCCAAGC-5′, | (SEQ ID NO:24) |
| 3′-CAGATTAGTAAGCGCGGCAGTT-5′, | (SEQ ID NO:25) |
| 3′-GGTCCTTATATCTTGCAATCCC-5′, | (SEQ ID NO:26) |
| 3′-CGGCCATCTCGATAATGGTAG-3′, | (SEQ ID NO:27) |
| 3′-GTCGTAACAGACTATTTCCGATACG-3′, | (SEQ ID NO:28) |
| 3′-CTATACCACCCTCGTCTATTTCC-5′, | (SEQ ID NO:29) |
| 3′-TCAGTACGACTACGCTAACGAG-5′, | (SEQ ID NO:30) |
| 3′-CGCTAGTCCCAGTTTTCCTAATGGT-5′, | (SEQ ID NO:31) |
| 3′-GCCTCGTACTACCAATAGGAC-3′, | (SEQ ID NO:32) |
| 3′-GTGGTCTATCATAAACACCTGCACC-3′, and | (SEQ ID NO:33) |
| 3′-TGGATACATCCCCAAGGTCGTT-3′. | (SEQ ID NO:34) |

The probe may comprise DNA, RNA, PNA or a derivative thereof. It may also comprise both DNA and RNA or derivatives thereof, and may additionally be labeled. The label can be a chemical, an enzymatic, an immunogenic, a radioactive, a fluorescent, a luminescent or a FRET label.

The present invention further includes an array of oligonucleotide probes immobilized on a support for detecting GBS, which array comprises a support suitable for use in nucleic acid hybridization having immobilized thereon a plurality of oligonucleotide probes, at least one of which is described above. The support may further comprise a surface that is selected from the group consisting of a silicon, a plastic, a glass, a ceramic, a rubber, and a polymer surface.

The present invention also provides a method for detecting GBS in a sample comprising the steps of: a) providing an oligonucleotide probe as described above; b) contacting the probe with a sample containing or suspected of containing a GBS target nucleotide sequence under conditions suitable for hybridization between the probe and the target nucleotide sequence; and c) assessing hybridization between the probe and the target nucleotide sequence to detect the GBS in said sample.

In any of the aforementioned methods, a plurality of samples can be assayed sequentially or simultaneously. The sample may be of human origin, but may also be sputum, urine, blood, tissue section, food, soil and water sample.

The methods described herein are useful for detecting any GBS.

### BRIEF DESCRIPTION OF THE DRAWINGS

FIG. 1 illustrates the nucleotide sequence of a cps gene; GenBank Accession Number AFB028896. The sequences of exemplary probes (SEQ ID NOs: 9 to 16) are underlined. The overlapping sequences of two of the probes (SEQ ID NOs:9 and 10) are shown by “_______ ”.

FIG. 2 illustrates the nucleotide sequence of a portion of a cps gene; GenBank Accession Number AF355776, nucleotides 15,241 to 16,020. The sequences of exemplary probes (SEQ ID NOs:17-21) are underlined.

FIG. 3 illustrates the specificity of AGT03009 (SEQ ID NO: 11) and AGT03011 (SEQ ID NO: 18), which distinguishes GBS DNA from Human and E. coli DNA.

### DETAILED DESCRIPTION OF THE INVENTION

The present invention provides cps probes that are specific for Streptococcus agalactiae and their use in diagnostic assays. The use of specific oligonucleotide sequences as probes in hybridization-based detection of infectious agents is becoming a valuable identification assay as an alternative to the problematic immuno-diagnostic and DNA amplification methods. The GBS-specific probes identified in this invention provide the basis for a complete “specimen to result” protocol using the probes with hybridization-based method and nucleic acid labeling method disclosed herein.

For clarity of disclosure, and not by way of limitation, the detailed description of the invention is divided into the subsections that follow.

Definitions

Unless defined otherwise, all technical and scientific terms used herein have the same meaning as is commonly understood by one of ordinary skill in the art to which this invention belongs. All patents, patent applications, and other publications and sequences from GenBank and other databases referred to herein are incorporated by reference in their entirety. If a definition set forth in this section is contrary to or otherwise inconsistent with a definition found in such incorporated references, the definition set forth in this section prevails over the definition that is incorporated herein by reference.

As used herein, “meningitis” refers to inflammation of the membranes of the brain or spinal cord.

As used herein, “Streptococcus” means a genus of nonmotile (with few exceptions), non-spore-forming, aerobic to facultatively anaerobic bacteria (family Lactobacillaceae) containing Gram-positive, spherical or ovoid cells which occur in pairs or short or long chains. Dextrorotatory lactic acid is the main product of carbohydrate fermentation. These organisms occur regularly in the mouth and intestines of humans and other animals, in dairy and other food products, and in fermenting plant juices.

As used herein, “Streptococcus agalactiae” or “S. agalactiae” or “GBS” refers to a species of Streptococcus found in the milk and tissues from udders of cows with mastitis; also reported to be associated with a variety of human infections, especially those of the urogenital tract.

As used herein, “primer” refers to an oligonucleotide that hybridizes to a target sequence, typically to prime the nucleic acid in the amplification process.

As used herein, “probe” refers to an oligonucleotide that hybridizes to a target sequence, typically to facilitate its detection, but which also may serve as a primer. The term “target sequence” refers to a nucleic acid sequence to which the probe specifically binds. Unlike a primer that is used to prime the target nucleic acid in the amplification process, a probe need not be extended to amplify target sequence using a polymerase enzyme. However, it will be apparent to those skilled in the art that probes and primers are structurally similar or identical in many cases.

As used herein, “complementary” means that two nucleic acid sequences have at least 50% sequence identity. Preferably, the two nucleic acid sequences have at least 60%, 70%, 80%, 90%, 95%, 96%, 97%, 98%, 99% or 100% sequence identity. “Complementary” also means that two nucleic acid sequences can hybridize under low, middle and/or high stringency condition(s).

As used herein, “substantially complementary” means that two nucleic acid sequences have at least 90% sequence identity. Preferably, the two nucleic acid sequences have at least 95%, 96%, 97%, 98%, 99% or 100% sequence identity. Alternatively, “substantially complementary” means that two nucleic acid sequences can hybridize under high stringency condition(s).

As used herein, “two perfectly matched nucleotide sequences” refers to a nucleic acid duplex wherein the two nucleotide strands match according to the Watson-Crick basepair principle, e.g., A-T and C-G pairs in DNA:DNA duplex and A-U and C-G pairs in DNA:RNA or RNA:RNA duplex, and there is no deletions or additions in either of the sequences in the duplex.

As used herein: “stringency of hybridization” in determining percentage mismatch is as follows:

1) high stringency: 0.1×SSPE, 0.1% SDS, 65° C.;

2) medium stringency: 0.2×SSPE, 0.1% SDS, 50° C. (also referred to as moderate stringency); and

3) low stringency: 1.0×SSPE, 0.1% SDS, 50° C.

It is understood that equivalent stringencies may be achieved using alternative buffers, salts and temperatures (See generally, Ausubel (Ed.) Current Protocols in Molecular Biology, 2.9A. Southern Blotting, 2.9B. Dot and Slot Blotting of DNA. and 2.10. Hybridization Analysis of DNA Blots, John Wiley & Sons, Inc. (2000)).

As used herein, “hairpin structure” refers to a nucleic acid that contains a double-stranded stem segment and a single-stranded loop segment wherein the two sides of the double-stranded stem segment are linked and separated by the single-stranded loop segment. The “hairpin structure” can also include 3′ and/or 5′ single-stranded region(s) extending from the double-stranded stem segment.

As used herein, “G+C content” refers to the percentage of the number of Gs (guanines) and Cs (cytoseines) in the nucleotide sequence, excluding poly T tails/spacers.

As used herein, a probe that “does not contain any hairpin secondary structure” means that the nucleotide sequence in the probe that is complementary to a target nucleotide sequence cannot form a hairpin structure within itself. However, the nucleotide sequence in the probe that is complementary to a target nucleotide sequence can be part of a nucleic acid that may form a hairpin structure under suitable conditions. For example, the nucleotide sequence complementary to a target nucleotide sequence can be located within a hairpin structure, or can be located at the junction of the loop and stem region of the hairpin structure.

As used herein, “melting temperature” (“Tm”) refers to the midpoint of the temperature range over which nucleic acid duplex, e.g., DNA:DNA, DNA:RNA and RNA:RNA, is denatured. The Tm of the probe herein means the Tm of the hybridized probe.

As used herein, “assessing” refers to quantitative and/or qualitative determination of the hybrid formed between the probe and the target nucleotide sequence, e.g., obtaining an absolute value for the amount or concentration of the hybrid, and also of obtaining an index, ratio, percentage, visual or other value indicative of the level of hybridization. Assessment may be direct or indirect, and the chemical species actually detected need not be the hybrid itself but may, for example, be a derivative thereof, reduction or disappearance of the probe and/or the target nucleotide sequence, or some further substance.

Probes for Detecting Streptococcus agalactiae

The present invention provides probes for detecting GBS that contain a nucleotide sequence that hybridizes with a target nucleotide sequence of a capsular polysaccharide synthesis (cps) gene. Exemplary GBS strains include, but are not limited to, NCS215, NCS915, NCS535, NCS246, COH1, C388/90, CNTC 1/82, CNCTC 1/82, and NT6. The probes can be in any suitable form. For example, the probe can comprise DNA, RNA, PNA or a derivative thereof. Alternatively, the probe can comprise both DNA and RNA or derivatives thereof. The probe can be single-stranded and be ready to be used in a hybridization analysis. Alternatively, the probe can be double-stranded and be denatured into single-stranded form prior to the hybridization analysis.

The oligonucleotide probes can be produced by any suitable method. For example, the probes can be chemically synthesized (See generally, Ausubel (Ed.) Current Protocols in Molecular Biology, 2.11. Synthesis and purification of oligonucleotides, John Wiley & Sons, Inc. (2000)), isolated from a natural source, produced by recombinant methods or a combination thereof. Synthetic oligonucleotides can be prepared by using the triester method of Matteucci et al., J. Am. Chem. Soc., 3: 3185-3191 (1981). Alternatively, automated synthesis maybe preferred, for example, on an Applied Biosynthesis DNA synthesizer using cyanoethyl phosphoramidate chemistry. Preferably, the probes are chemically synthesized.

Suitable bases for preparing the oligonucleotide probes of the present invention may be selected from naturally occurring nucleotide bases such as adenine, cytosine, guanine, uracil, and thymine. It may also be selected from non-naturally occurring or “synthetic” nucleotide bases such as 8-oxo-guanine, 6-mercaptoguanine, 4-acetylcytidine, 5-(carboxyhydroxyethyl) uridine, 2′-O-methylcytidine, 5-carboxymethylamino-methyl-2-thiouridine, 5-carboxymethylaminomethyl uridine, dihydrouridine, 2′-O-methylpseudouridine, beta-D-galactosylqueosine, 2′-O-methylguanosine, inosine, N⁶-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N⁶-methyladenosine, 7-methylguanosine, 5-methylaminomethyluridine, 5-methoxyaminomethyl-2-thiouridine, beta-D-mannosylqueosine, 5-methoxycarbonylmethyluridine, 5-methoxyuridine, 2-methylthio-N⁶-isopentenyladenosine, N-((9-.beta.-D-ribofuranosyl-2-methylthiopurine-6-yl)carbamoyl)threonine, N-((9-beta-D-ribofuranosylpurine-6-yl) N-methylcarbamoyl) threonine, uridine-5-oxyacetic acid methylester, uridine-5-oxyacetic acid, wybutoxosine, pseudouridine, queosine, 2-thiocytidine, 5-methyl-2-thiouridine, 2-thiouridine, 2-thiouridine, 5-methyluridine, N-((9-beta-D-ribofuranosylpurine-6-yl) carbamoyl) threonine, 2′-O-methyl-5-methyluridine, 2′-O-methyluridine, wybutosine, and 3-(3-amino-3-carboxypropyl) uridine.

Likewise, chemical analogs of oligonucleotides (e.g., oligonucleotides in which the phosphodiester bonds have been modified, e.g., to the methylphosphonate, the phosphotriester, the phosphorothioate, the phosphorodithioate, or the phosphoramidate) may also be employed. Protection from degradation can be achieved by use of a “3′-end cap” strategy by which nuclease-resistant linkages are substituted for phosphodiester linkages at the 3′ end of the oligonucleotide (Shaw et al., Nucleic Acids Res. 19: 747-50 (1991)). Phosphoramidates, phosphorothioates, and methylphosphonate linkages all function adequately in this manner. More extensive modification of the phosphodiester backbone has been shown to impart stability and may allow for enhanced affinity and increased cellular permeation of oligonucleotides (Milligan et al., J. Med. Chem. 36: 1923-37 (1993)). Many different chemical strategies have been employed to replace the entire phosphodiester backbone with novel linkages. Backbone analogues include phosphorothioate, phosphorodithioate, methylphosphonate, phosphoramidate, boranophosphate, phosphotriester, formacetal, 3′-thioformacetal, 5′-thioformacetal, 5′-thioether, carbonate, 5′-N-carbamate, sulfate, sulfonate, sulfamate, sulfonamide, sulfone, sulfite, sulfoxide, sulfide, hydroxylamine, methylene (methylimino) (MMI) or methyleneoxy (methylimino) (MOMI) linkages. Phosphorothioate and methylphosphonate-modified oligonucleotides are particularly preferred due to their availability through automated oligonucleotide synthesis. The oligonucleotide may be a “peptide nucleic acid” such as described by Nielsen et al., Science 254: 1497 (1991). The only requirement is that the oligonucleotide probe should possess a sequence at least a portion of which is capable of binding to a portion of the sequence of a target DNA molecule.

Hybridization probes can be of any suitable length. There is no lower or upper limits to the length of the probe, as long as the probe hybridizes to the target cps nucleic acid sequence(s) and functions effectively as a probe (e.g., facilitates detection). The probes of the present invention can be as short as 50, 40, 30, 20, 15, 10 or 8 nucleotides, or shorter. Likewise, the probes can be as long as 20, 40, 50, 60, 75, 100 or 200 nucleotides, or longer, e.g., to the full length of the cps sequence. Generally, the probes will have at least 8 nucleotides, preferably at least 14 nucleotides, more preferably at least 19 nucleotides, and will not contain any hairpin secondary structures. In specific embodiments, the probe can have a length of at least 30 nucleotides or at least 50 nucleotides. If there is to be complete complementarity, e.g., if the target strand contains a sequence identical to that of the probe, the duplex will be relatively stable under even stringent conditions and the probes may be short, e.g., in the range of about 10-30 base pairs. If some degree of mismatch is expected in the probe, e.g., if it is suspected that the probe would hybridize to a variant region, or to a group of sequences such as all species within a specific genus, e.g., Streptococcus species, the probe may be of greater length (e.g., 15-40 bases) to balance the effect of the mismatch(es).

The probe should have a G+C content ranging from about 30% to about 70%. Preferably, the probe has G+C content ranging from about 55% to about 65%. The probe should have a Tm value ranging from about 40° C. to about 80° C., preferably from about 60° C. to about 75° C.

The probes used in the present invention are selected to be “substantially complementary” to the different strands of each specific sequence to be hybridized. The probes need not reflect the exact sequence of the template, but must be sufficiently complementary to hybridize selectively with their respective strands. Non-complementary bases or longer sequences can be interspersed into the probe, provided that the probe retains sufficient complementarity with the sequence of one of the strands to be hybridized to form a duplex structure which can be detected. The non-complementary nucleotide sequences of the probes may include restriction enzyme sites. Appending a restriction enzyme site to the end(s) of the target sequence is particularly helpful for subsequent cloning of the target sequence.

The probe should also be specific for GBS. In other words, it should be capable of differentiating GBS fro other Streptococci species. Any suitable contiguous nucleotide sequence that is at least 8 nucleotides in length, or a complementary strand thereof, within the cps subsequences (SEQ ID NOs:2-8) can be used as a target nucleotide sequence as follows:

(SEQ ID NO:2)

5′-AACCGACCTTGATGAAGAAGTGAAATCGACCATCATTCAATTAAGAC
AGTTCGAAAAATCAGTCA GAAATCCTTT-3′
(GenBank AFB028896 125-200)

(SEQ ID NO:3)

5′-TAGACAATATCAATAACATCATCCACGTCAAGAGGGATTGCGACAAT
TTCGTCACCGTTCAATTTTGAGTTGAGGATACCAGTTGCCACTGTATAAC
CATCTAATCCAATCATTAAATTAAACAGAGTTGCTCTATCACTGACAACA
ATGGATTTTGGGTGAGGAATTTGAGACATCATTTCCTCAGAAAAGTAGAA
AGAA-3′
(GenBank AFB028896 500-700)

(SEQ ID NO:4)

5′-TAATAGCATAAGAAATTATGAATATTTAGACAAAGTGATACCAGTTG
AAGAAGCACTACTAAACATGTATGACATTAAAACTTATGGTTCAATTTTT
ATTACTGCATGGGGAAAATTATTCCATAAATCTATATTCAATGATTTAGA
ATTTGCATTAAATAAGTATCATGAGGATGAATTCTTTAACTATAAAGCAT
ACTTAAAAGCTAATTCTATAACATACATAGACAAGCCTCTCTATCATTAT
CGTATACGAGTAGGTAGTATCATGAATAATAGTGATAATGTTATAATTGC
TAGAAAGAAACTTG ATGTTTTATCAGCATTAGACGAGCGAATAA-3′
(GenBank AFB028896 10,100-10,440)

(SEQ ID NO:5)

5′-TCCAGTGTCTATTTATAGGATCCAATTCAGATACTGGTTCTGATGAT
ATTGCAAAGGCTATCAATACTTATTTGATAAATCATGAAAATAGTTATTG
CTTTGCTTCTTTATCAACCCAACTTTATCATTCCTTAATTCGTCATTCAC
TAGGACTGATTGGTAATTCGTCTTCTGGTTTAATAGAAGTTCCTTCTTTG
ATGAAACCTACTCTTAATATTGGTGATCGCCAAAAAGGTAGGCTACATGG
AGAA-3′
(GenBank AFB028896 14,900-15,150)

(SEQ ID NO:6)

5′-TCTAAAATGAAAAAAAAGTTGGCATTTTTAGGCGCAGGGAGTCATGC
TGATGCGATTGCTCCGGTTATTGATCCTCTTGTGTATGATTTCGTAGGTT
TTTTTGATGATAAAGATATTACGGAGCATGATGGTTATCCTGTTCTTGGA
AAACTTTATGATGTGCTACCTTACCTTGA-3′
(GenBank AF355776 15,300-15,475)

(SEQ ID NO:7)

5′-GTATTGACACCAGATAGTATTTGTGGACGTGGTATCTTTATTGGTTT
TGGGGCTTTTATAGGTTCTAAAGTGAAGCTGTTTGATAACAACGTTGTTA
ATAC-3′
(GenBank AF355776 15,600-15,700)

(SEQ ID NO:8)

5′-CGACAATTGGAGCAGGAGCTGTTGTTGTAAAAGATATTATAGAACCT
GGAACCTATGTAGGGGTTCCAGCAAAAAAAATAAAATAAAGGATATGTTA
TGAAGCCAATTTGTATTATTCCTGCGCGATCAGGGTCAAAAGGATTACCA
GATA-3′
(GenBank AF355776 15,850-16,000)

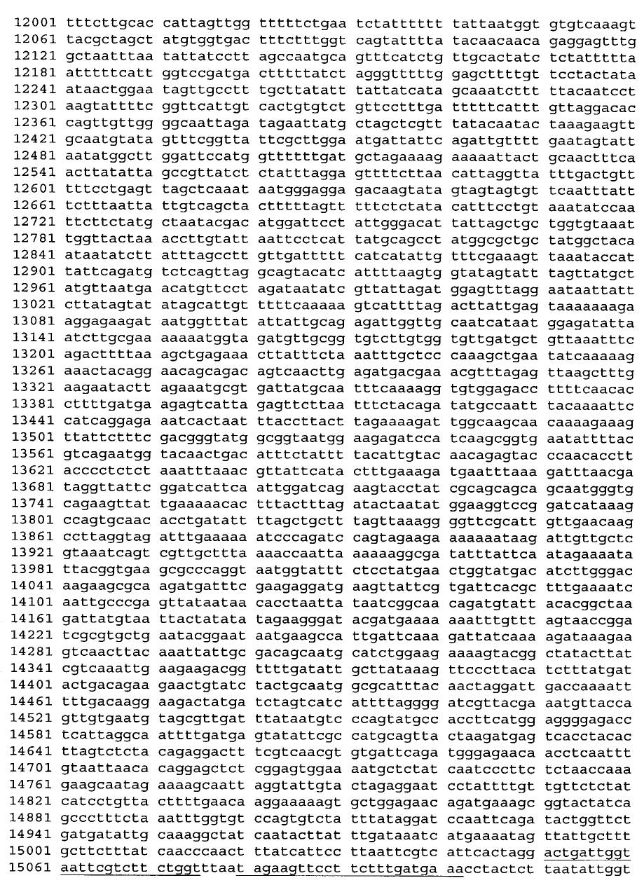

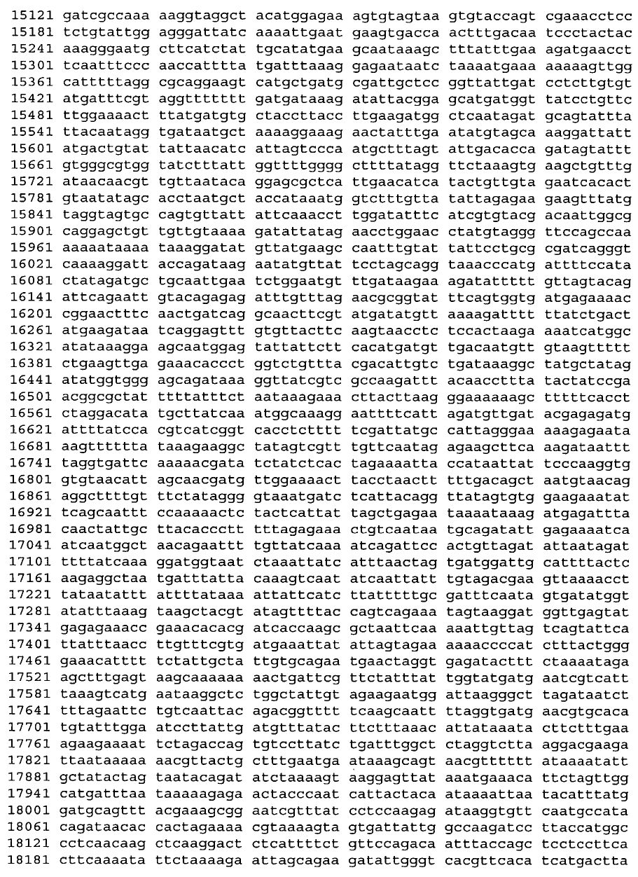

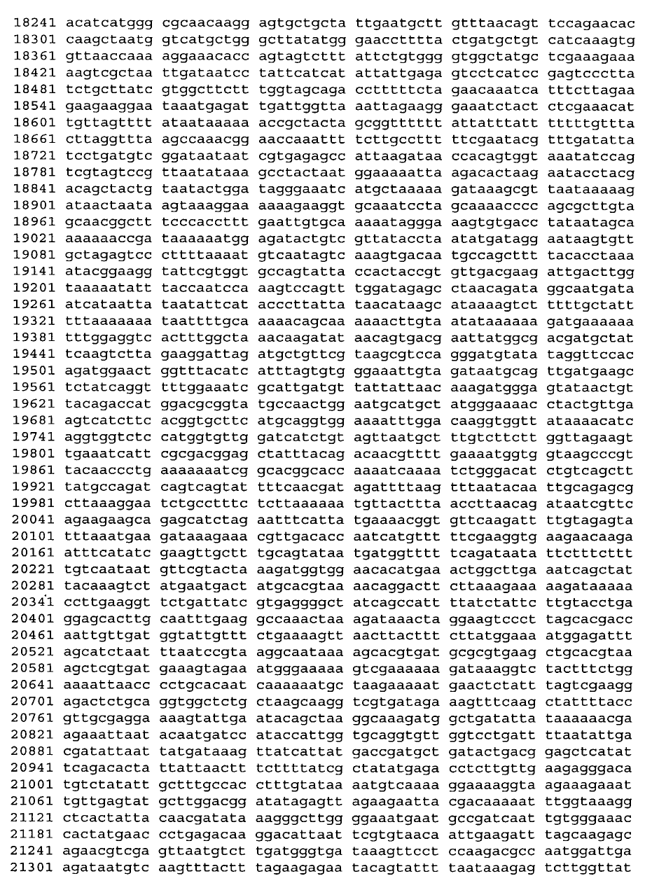

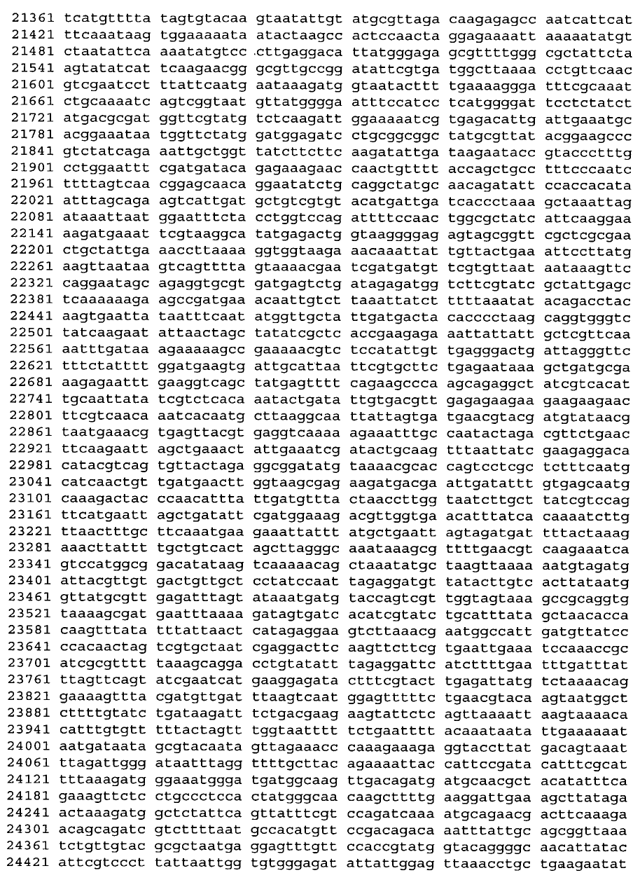

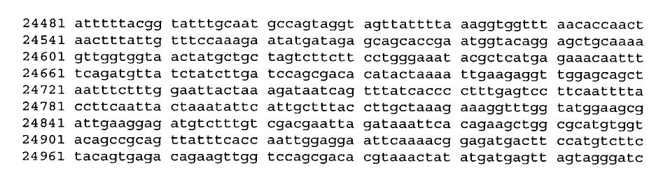

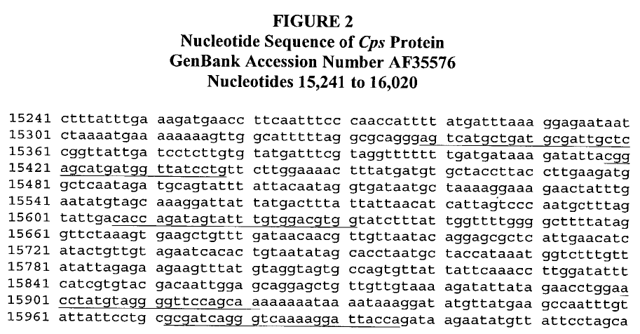

In one embodiment, the target sequence comprises the NeuD gene, which consists, e.g., of a 630 bp segment of GenBank Accession No. AF355776, from nucleotide 15,290 to 15,935 (See FIG. 2) (SEQ. ID NO: 35.) In another embodiment, the target sequence comprises the NeuA gene, which consists, e.g., of a 1,251 bp segment of GenBank Accession No. AF35576, from nucleotide 15,936 to 17,187. A particularly preferred target sequence spans both the NeuA and NeuD gene sequences, from nucleotide 15,850 to 16,000 (SEQ ID NO:8.)

Typically, the probes of the present invention hybridize to consecutive nucleotides of the cps sequence under stringent conditions, as defined elsewhere. Alternatively stated, probes of the present invention will be at least 75%, 80%, 85%, 90% or even 95% homologous or more with consecutive nucleotides within the cps sequence, or complementary sequences thereof.

Preferably, the present invention provides for an oligonucleotide probe for detecting GBS in a sample comprising a nucleotide sequence that hybridizes with a target nucleotide sequence, wherein the target nucleotide sequence is all or part of the cps gene, or a complementary strand thereof, and wherein the probe has a G+C content from about 30 to 70%, a Tm value from about 40 to 80° C., a length of at least 8 nucleotides (if DNA and/or RNA, or 6 if PNA or other nucleic acid analog with a higher affinity than DNA or RNA) and does not contain any hairpin secondary structure. Preferably, the probe hybridizes with a target nucleotide sequence of a Streptococcus agalactiae cps gene under middle or high stringency.

The sequences of exemplary probes are:

| | |
|---|---|
| 5′-GCCGCACCAGTTAAAAACGATAC-3′, | (SEQ ID NO:9; a.k.a. AGT03007) |
| 5′-TGTCAGCAAATTAGCCGCACCAGT-3′, | (SEQ ID NO:10; a.k.a. AGT03008) |
| 5′-GAGTTGATGTCAACAGGTCGGTTCG-3′, | (SEQ ID NO:11; a.k.a. AGT03009) |
| 5′-GTCTAATCATTCGCGCCGTCAA-3′, | (SEQ ID NO:12; a.k.a. Sagal-cps-03) |
| 5′-CCAGGAATATAGAACGTTAGGG-3′, | (SEQ ID NO:13; a.k.a. Sagal-cps-04) |
| 5′-GCCGGTAGAGCTATTACCATC-3′. | (SEQ ID NO:14; a.k.a. Sagal-cps-05) |
| 5′-CAGCATTGTCTGATAAAGGCTATGC-3′, | (SEQ ID NO:15; a.k.a. Sagal-cps-07) |
| 5′-GATATGGTGGGAGCAGATAAAGG-3′, | (SEQ ID NO:16; a.k.a. Sagal-cps-08) |
| 5′-AGTCATGCTGATGCGATTGCTC-3′, | (SEQ ID NO:17; a.k.a. AGT03010) |
| 5′-GCGATCAGGGTCAAAAGGATTACCA-3′, | (SEQ ID NO:18; a.k.a. AGT03011) |
| 5′-CGGAGCATGATGGTTATCCTG-3′, | (SEQ ID NO:19; a.k.a. Sagal-cps-neuD02) |
| 5′-CACCAGATAGTATTTGTGGACGTGG-3′, and | (SEQ ID NO:20; a.k.a. Sagal-cps-neuD03) |
| 5′-ACCTATGTAGGGGTTCCAGCAA-3′. | (SEQ ID NO:21; a.k.a. Sagal-cps-neuD04) |

Immobilization of Probes

The present invention provides a method for detecting GBS in a sample comprising the steps of: a) providing an oligonucleotide probe as described above; b) contacting the probe provided in step a) with a sample suspected of containing a GBS target nucleotide sequence under conditions suitable for hybridization between the probe and the target nucleotide sequence; and c) assessing hybridization between the probe and the target nucleotide sequence to detect GBS in the sample. The present invention also provides an array of immobilized oligonucleotide probes for detecting (GBS).

The present method can be used in solution. Preferably, it is conducted in chip format, e.g., by using the probe(s) immobilized on a solid support.

The probes can be immobilized on any suitable surface, preferably, a solid support, such as silicon, plastic, glass, ceramic, rubber, or polymer surface. The probe may also be immobilized in a 3-dimensional porous gel substrate, e.g., Packard HydroGel chip (Broude et al., Nucleic Acids Res., 29(19):E92 (2001)).

For an array-based assay, the probes are preferably immobilized to a solid support such as a “biochip”. The solid support may be biological, nonbiological, organic, inorganic, or a combination of any of these, existing as particles, strands, precipitates, gels, sheets, tubing, spheres, containers, capillaries, pads, slices, films, plates, slides, etc.

A microarray biochip containing a library of probes can be prepared by a number of well known approaches including, for example, light-directed methods, such as VLSIPS™ described in U.S. Pat. Nos. 5,143, 854, 5,384,261 or 5,561,071; bead based methods such as described in U.S. Pat. No. 5,541,061; and pin based methods such as detailed in U.S. Pat. No. 5,288,514. U.S. Pat. No. 5,556,752, which details the preparation of a library of different double stranded probes as a microarray using the VLSIPS™, is also suitable for preparing a library of hairpin probes in a microarray.

Flow channel methods, such as described in U.S. Pat. Nos. 5,677,195 and 5,384,261, can be used to prepare a microarray biochip having a variety of different probes. In this case, certain activated regions of the substrate are mechanically separated from other regions when the probes are delivered through a flow channel to the support. A detailed description of the flow channel method can be found in U.S. Pat. No. 5,556,752, including the use of protective coating wetting facilitators to enhance the directed channeling of liquids though designated flow paths.

Spotting methods also can be used to prepare a microarray biochip with a variety of probes immobilized thereon. In this case, reactants are delivered by directly depositing relatively small quantities in selected regions of the support. In some steps, of course, the entire support surface can be sprayed or otherwise coated with a particular solution. In particular formats, a dispenser moves from region to region, depositing only as much probe or other reagent as necessary at each stop. Typical dispensers include micropipettes, nanopippettes, ink-jet type cartridges and pins to deliver the probe containing solution or other fluid to the support and, optionally, a robotic system to control the position of these delivery devices with respect to the support. In other formats, the dispenser includes a series of tubes or multiple well trays, a manifold, and an array of delivery devices so that various reagents can be delivered to the reaction regions simultaneously. Spotting methods are well known in the art and include, for example, those described in U.S. Pat. Nos. 5,288,514, 5,312,233 and 6,024,138. In some cases, a combination of flow channels and “spotting” on predefined regions of the support also can be used to prepare microarray biochips with immobilized probes.

A solid support for immobilizing probes is preferably flat, but may take on alternative surface configurations. For example, the solid support may contain raised or depressed regions on which probe synthesis takes place or where probes are attached. In some embodiments, the solid support can be chosen to provide appropriate light-absorbing characteristics. For example, the support may be a polymerized Langmuir Blodgett film, glass or functionalized glass, Si, Ge, GaAs, GaP, SiO₂, SiN₄, modified silicon, or any one of a variety of gels or polymers such as (poly)tetrafluoroethylene, (poly)vinylidendifluoride, polystyrene, polycarbonate, or combinations thereof. Other suitable solid support materials will be readily apparent to those of skill in the art.

The surface of the solid support can contain reactive groups, which include carboxyl, amino, hydroxyl, thiol, or the like, suitable for conjugating to a reactive group associated with an oligonucleotide or a nucleic acid. Preferably, the surface is optically transparent and will have surface Si—OH functionalities, such as those found on silica surfaces.

The probes can be attached to the support by chemical or physical means such as through ionic, covalent or other forces well known in the art. Immobilization of nucleic acids and oligonucleotides can be achieved by any means well known in the art (see, e.g., Dattagupta et al., Analytical Biochemistry, 177:85-89(1989); Saiki et al., Proc. Natl. Acad. Sci. USA, 86:6230-6234(1989); and Gravitt et al., J. Clin. Micro., 36:3020-3027(1998)).

The probes can be attached to a support by means of a spacer molecule, e.g., as described in U.S. Pat. No. 5,556,752 to Lockhart et al., to provide space between the double stranded portion of the probe as may be helpful in hybridization assays. A spacer molecule typically comprises between 6-50 atoms in length and includes a surface attaching portion that attaches to the support. Attachment to the support can be accomplished by carbon-carbon bonds using, for example, supports having (poly)trifluorochloroethylene surfaces, or preferably, by siloxane bonds (using, for example, glass or silicon oxide as the solid support). Siloxane bonding can be formed by reacting the support with trichlorosilyl or trialkoxysilyl groups of the spacer. Aminoalkylsilanes and hydroxyalkylsilanes, bis(2-hydroxyethyl)-aminopropyltriethoxysilane, 2-hydroxyethylaminopropyltriethoxysilane, aminopropyltriethoxysilane or hydroxypropyltriethoxysilane are useful are surface attaching groups.

The spacer can also include an extended portion or longer chain portion that is attached to the surface-attaching portion of the probe. For example, amines, hydroxyl, thiol, and carboxyl groups are suitable for attaching the extended portion of the spacer to the surface-attaching portion. The extended portion of the spacer can be any of a variety of molecules which are inert to any subsequent conditions for polymer synthesis. These longer chain portions will typically be aryl acetylene, ethylene glycol oligomers containing 2-14 monomer units, diamines, diacids, amino acids, peptides, or combinations thereof.

In some embodiments, the extended portion of the spacer is a polynucleotide or the entire spacer can be a polynucleotide. The extended portion of the spacer also can be constructed of polyethyleneglycols, polynucleotides, alkylene, polyalcohol, polyester, polyamine, polyphosphodiester and combination s thereof. Additionally, for use in synthesis of probes, the spacer can have a protecting group attached to a functional group (e.g., hydroxyl, amino or carboxylic acid) on the distal or terminal end of the spacer (opposite the solid support). After deprotection and coupling, the distal end can be covalently bound to an oligomer or probe.

The present method can be used to analyze a single sample with a single probe at a time. Preferably, the method is conducted in high-throughput format. For example, a plurality of samples can be analyzed with a single probe simultaneously, or a single sample can be analyzed using a plurality of probes simultaneously. More preferably, a plurality of samples can be analyzed using a plurality of probes simultaneously.

In a specific embodiment, the probe or the GBS target nucleotide sequence is immobilized on a solid support. In another specific embodiment, a plurality of the probes immobilized on a solid support is used. In still another specific embodiment, a plurality of samples is assayed. Preferably, the plurality of samples is assayed simultaneously.

Hybridization Conditions

Hybridization can be carried out under any suitable technique known in the art. It will be apparent to those skilled in the art that hybridization conditions can be altered to increase or decrease the degree of hybridization, the level of specificity of the hybridization, and the background level of non-specific binding (e.g., by altering hybridization or wash salt concentrations or temperatures). The hybridization between the probe and the target nucleotide sequence can be carried out under any suitable stringencies, including high, middle or low stringency. Typically, hybridizations will be performed under conditions of high stringency.

The hybridization can be carried out at any suitable temperature. For example, if the present probe is part of a hairpin structure, the oligonucleotide probe and the target nucleotide sequence can be contacted at a temperature from about 4° C. to about 90° C. Preferably, the oligonucleotide probe and the target nucleotide sequence can be contacted at a temperature from about 40° C. to about 80° C., preferably from about 60° C. to about 75° C.

In addition, the hybridization can be carried out for any suitable period of time. For example, if the present probe is part of a hairpin structure as disclosed in co-owned PCT Patent Application No. WO 02/106531, the oligonucleotide probe and the target nucleotide sequence can be contacted for a time from about 1 minute to about 60 minutes. Preferably, the oligonucleotide probe and the target nucleotide sequence can be contacted for a time from about 15 minutes to about 30 minutes.

Hybridization between the probe and target nucleic acids can be homogenous, e.g., typical conditions used in molecular beacons (Tyagi S. et al., Nature Biotechnology, 14:303-308 (1996); and U.S. Pat. No. 6,150,097) and in hybridization protection assay (Gen-Probe, Inc.) (U.S. Pat. No. 6, 004,745), or heterogeneous (typical conditions used in different type of nitrocellulose based hybridization and those used in magnetic bead based hybridization).

The target polynucleotide sequence may be detected by hybridization with an oligonucleotide probe that forms a stable hybrid with that of the target sequence under high to low stringency hybridization and wash conditions. An advantage of detection by hybridization is that, depending on the probes used, additional specificity is possible. If it is expected that the probes will be completely complementary (e.g., about 99% or greater) to the target sequence, high stringency conditions will be used. If some mismatching is expected, for example, if variant strains are expected with the result that the probe will not be completely complementary, the stringency of hybridization may be lessened. However, conditions are selected to minimize or eliminate nonspecific hybridization.

Species-specific hybridization of a target sequence refers to hybridization of a target sequence in the GBS, but little or no hybridization in non-GBS Streptococci. The probes that are disclosed herein hybridize to cps nucleic acids. Typically, the probes of the present invention hybridize to consecutive nucleotides of the cps sequences under stringent conditions, as defined below. Alternatively stated, probes of the present invention will be at least 75%, 80%, 85%, 90% or even 95% homologous or more with consecutive nucleotides within the cps sequences, in particular the probes given by SEQ. ID NOs: 9 to 21, or complementary strands thereof, which are given by SEQ ID NOs: 22-34, respectively. As nucleic acids do not require complete homology to hybridize, it will be apparent to those skilled in the art that the probe sequences specifically disclosed herein may be modified so as to be substantially homologous to the probe sequences disclosed herein without loss of utility as GBS probes.

Stringency requirements can be modified to alter target specificity as described. For example, where GBS is to be detected, it is well within the scope of the invention for those of ordinary skill in the art to modify the stringency conditions described above and cause other non-GBS to be excluded or included as targets. The new cps probes provided herein give particular hybridization characteristics as desired.

Conditions those affect hybridization and those select against nonspecific hybridization are known in the art (Molecular Cloning: A Laboratory Manual, second edition, J. Sambrook, E. Fritsch, T. Maniatis, Cold Spring Harbor Laboratory Press, 1989). Generally, lower salt concentration and higher temperature increase the stringency of hybridization. For example, in general, stringent hybridization conditions include incubation in solutions that contain approximately 0.1× SSC, 0.1% SDS, at about 65° C. incubation/wash temperature. Middle stringent conditions are incubation in solutions that contain approximately 1-2× SSC, 0.1% SDS and about 50° C.-65° C. incubation/wash temperature. The low stringency conditions are 2× SSC and about 30° C.-50° C.

An alternate method of hybridization and washing is first to carry out a low stringency hybridization (5× SSPE, 0.5% SDS) followed by a high stringency wash in the presence of 3M tetramethyl-ammonium chloride (TMAC). The effect of the TMAC is to equalize the relative binding of A-T and G-C base pairs so that the efficiency of hybridization at a given temperature corresponds more closely to the length of the polynucleotide. Using TMAC, it is possible to vary the temperature of the wash to achieve the level of stringency desired (Wood et al., Proc. Natl. Acad. Sci. USA, 82:1585-1588 (1985)).

A hybridization solution may contain 25% formamide, 5× SSC, 5× Denhardt's solution, 100 μg/ml of single stranded DNA, 5% dextran sulfate, or other reagents known to be useful for probe hybridization.

Detection of the Hybrid

Detection of hybridization between the probe and the GBS nucleic acids can be carried out by any method known in the art, e.g., labeling the probe, the secondary probe, the target nucleic acids or some combination thereof, and are suitable for purposes of the present invention. Alternatively, the hybrid may be detected by mass spectroscopy in the absence of detectable label (e.g., U.S. Pat. No. 6,300,076).

The detectable label is a moiety that can be detected either directly or indirectly after the hybridization. In other words, a detectable label has a measurable physical property (e.g., fluorescence or absorbance) or is participant in an enzyme reaction. Using direct labeling, the target nucleotide sequence or the probe is labeled, and the formation of the hybrid is assessed by detecting the label in the hybrid. Using indirect labeling, a secondary probe is labeled, and the formation of the hybrid is assessed by the detection of a secondary hybrid formed between the secondary probe and the original hybrid.

Methods of labeling probes or nucleic acids are well known in the art. Suitable labels include fluorophores, chromophores, luminophores, radioactive isotopes, electron dense reagents, FRET(fluorescence resonance energy transfer), enzymes and ligands having specific binding partners. Particularly useful labels are enzymatically active groups such as enzymes (Wisdom, Clin. Chem., 22:1243 (1976)); enzyme substrates (British Pat. No. 1,548,741); coenzymes (U.S. Pat. Nos. 4,230,797 and 4,238,565) and enzyme inhibitors (U.S. Pat. No. 4,134,792); fluorophores (Soini and Hemmila, Clin. Chem., 25:353 (1979)); chromophores including phycobiliproteins, luminescers such as chemiluminescers and bioluminescers (Gorus and Schram, Clin. Chem., 25:512 (1979) and ibid, 1531); specifically bindable ligands, e.g., protein binding ligands; antigens; and residues comprising radioisotopes such as ³H, ³⁵S, ³²P, ¹²⁵I, and ¹⁴C. Such labels are detected on the basis of their own physical properties (e.g., fluorescers, chromophores and radioisotopes) or their reactive or binding properties (e.g., antibodies, enzymes, substrates, coenzymes and inhibitors). Ligand labels are also useful for solid phase capture of the oligonucleotide probe (e.g., capture probes). Exemplary labels include biotin (detectable by binding to labeled avidin or streptavidin) and enzymes, such as horseradish peroxidase or alkaline phosphatase (detectable by addition of enzyme substrates to produce a colored reaction product).

For example, a radioisotope-labeled probe or target nucleic acid can be detected by autoradiography. Alternatively, the probe or the target nucleic acid labeled with a fluorescent moiety can detected by fluorimetry, as is known in the art. A hapten or ligand (e.g., biotin) labeled nucleic acid can be detected by adding an antibody or an antibody pigment to the hapten or a protein that binds the labeled ligand (e.g., avidin).

As a further alternative, the probe or nucleic acid may be labeled with a moiety that requires additional reagents to detect the hybridization. If the label is an enzyme, the labeled nucleic acid, e.g., DNA, is ultimately placed in a suitable medium to determine the extent of catalysis. For example, a cofactor-labeled nucleic acid can be detected by adding the enzyme for which the label is a cofactor and a substrate for the enzyme. Thus, if the enzyme is a phosphatase, the medium can contain nitrophenyl phosphate and one can monitor the amount of nitrophenol generated by observing the color. If the enzyme is a beta-galactosidase, the medium can contain o-nitro-phenyl-D-galacto-pyranoside, which also liberates nitrophenol. Exemplary examples of the latter include, but are not limited to, beta-galactosidase, alkaline phosphatase, papain and peroxidase. For in situ hybridization studies, the final product of the substrate is preferably water insoluble. Other labels, e.g., dyes, will be evident to one having ordinary skill in the art.

The label can be linked directly to the DNA binding ligand, e.g., acridine dyes, phenanthridines, phenazines, furocoumarins, phenothiazines and quinolines, by direct chemical linkage such as involving covalent bonds, or by indirect linkage such as by the incorporation of the label in a microcapsule or liposome, which in turn is linked to the binding ligand. Methods by which the label is linked to a DNA binding ligand such as an intercalator compound are well known in the art and any convenient method can be used. Representative intercalating agents include mono- or bis-azido aminoalkyl methidium or ethidium compounds, ethidium monoazide ethidium diazide, ethidium dimer azide (Mitchell et al., J. Am. Chem. Soc., 104:4265 (1982)), 4-azido-7-chloroquinoline, 2-azidofluorene, 4′-aminomethyl-4,5′-dimethylangelicin, 4′-aminomethyl-trioxsalen (4′aminomethyl-4,5′,8-trimethyl-psoralen), 3-carboxy-5- or -8-amino- or -hydroxy-psoralen. A specific nucleic acid binding azido compound has been described by Forster et al., Nucleic Acid Res., 13:745 (1985). Other useful photoreactable intercalators are the furocoumarins which form (2+2) cycloadducts with pyrimidine residues. Alkylating agents also can be used as the DNA binding ligand, including, for example, bis-chloroethylamines and epoxides or aziridines, e.g., aflatoxins, polycyclic hydrocarbon epoxides, mitomycin and norphillin A. Particularly useful photoreactive forms of intercalating agents are the azidointercalators. Their reactive nitrenes are readily generated at long wavelength ultraviolet or visible light and the nitrenes of arylamides prefer insertion reactions over their rearrangement products (White et al., Meth. Enzymol., 46:644 (1977)).

The probe may also be modified for use in a specific format such as the addition of 10-100 T residues for reverse dot blot or the conjugation to bovine serum albumin or immobilization onto magnetic beads.

When detecting hybridization by an indirect detection method, a detectably labeled second probe(s) can be added after initial hybridization between the probe and the target or during hybridization of the probe and the target. Optionally, the hybridization conditions may be modified after addition of the secondary probe. After hybridization, unhybridized secondary probe can be separated from the initial probe, for example, by washing if the initial probe is immobilized on a solid support. In the case of a solid support, detection of label bound to locations on the support indicates hybridization of a target nucleotide sequence in the sample to the probe.

The detectably labeled secondary probe can be a specific probe. Alternatively, the detectably labeled probe can be a degenerate probe, e.g., a mixture of sequences such as whole genomic DNA essentially as described in U.S. Pat. No. 5,348,855. In the latter case, labeling can be accomplished with intercalating dyes if the secondary probe contains double stranded DNA. Preferred DNA-binding ligands are intercalator compounds such as those described above.

A secondary probe also can be a library of random nucleotide probe sequences. The length of a secondary probe should be decided in view of the length and composition of the primary probe or the target nucleotide sequence on the solid support that is to be detected by the secondary probe. Such a probe library is preferably provided with a 3′ or 5′ end labeled with photoactivatable reagent and the other end loaded with a detection reagent such as a fluorophore, enzyme, dye, luminophore, or other detectably known moiety.

The particular sequence used in making the labeled nucleic acid can be varied. Thus, for example, an amino-substituted psoralen can first be photochemically coupled with a nucleic acid, the product having pendant amino groups by which it can be coupled to the label, e.g., labeling is carried out by photochemically reacting a DNA binding ligand with the nucleic acid in the test sample. Alternatively, the psoralen can first be coupled to a label such as an enzyme and then to the nucleic acid.

Advantageously, the DNA binding ligand is first combined with label chemically and thereafter combined with the nucleic acid probe. For example, since biotin carries a carboxyl group, it can be combined with a furocoumarin by way of amide or ester formation without interfering with the photochemical reactivity of the furocoumarin or the biological activity of the biotin. Aminomethylangelicin, psoralen and phenanthridium derivatives can similarly be linked to a label, as can phenanthridium halides and derivatives thereof such as aminopropyl methidium chloride (Hertzberg et al, J. Amer. Chem. Soc., 104:313 (1982)). Alternatively, a bifunctional reagent such as dithiobis succinimidyl propionate or 1,4-butanediol diglycidyl ether can be used directly to couple the DNA binding ligand to the label where the reactants have alkyl amino residues, again in a known manner with regard to solvents, proportions and reaction conditions. Certain bifunctional reagents, possibly glutaraldehyde may not be suitable because, while they couple, they may modify nucleic acid and thus interfere with the assay. Routine precautions can be taken to prevent such difficulties.

Also advantageously, the DNA binding ligand can be linked to the label by a spacer, which includes a chain of up to about 40 atoms, preferably about 2 to 20 atoms, including, but not limited to, carbon, oxygen, nitrogen and sulfur. Such spacer can be the polyfunctional radical of a member including, but not limited to, peptide, hydrocarbon, polyalcohol, polyether, polyamine, polyimine and carbohydrate, e.g., -glycyl-glycyl-glycyl- or other oligopeptide, carbonyl dipeptides, and omega-amino-alkane-carbonyl radical or the like. Sugar, polyethylene oxide radicals, glyceryl, pentaerythritol, and like radicals also can serve as spacers. Spacers can be directly linked to the nucleic acid-binding ligand and/or the label, or the linkages may include a divalent radical of a coupler such as dithiobis succinimidyl propionate, 1,4-butanediol diglycidyl ether, a diisocyanate, carbodiimide, glyoxal, glutaraldehyde, or the like.

Secondary probe for indirect detection of hybridization can be also detected by energy transfer such as in the “beacon probe” method described by Tyagi and Kramer, Nature Biotech., 14:303-309 (1996) or U.S. Pat. Nos. 5,119,801 and 5,312,728 to Lizardi et al. Any FRET detection system known in the art can be used in the present method. For example, the AlphaScreen™ system can be used. AlphaScreen technology is an “Amplified Luminescent Proximity Homogeneous Assay” method. Upon illumination with laser light at 680 nm, a photosensitizer in the donor bead converts ambient oxygen to singlet-state oxygen. The excited singlet-state oxygen molecules diffuse approximately 250 nm (one bead diameter) before rapidly decaying. If the acceptor bead is in close proximity of the donor bead, by virtue of a biological interaction, the singlet-state oxygen molecules reacts with chemiluminescent groups in the acceptor beads, which immediately transfer energy to fluorescent acceptors in the same bead. These fluorescent acceptors shift the emission wavelength to 520-620 nm. The whole reaction has a 0.3 second half-life of decay, so measurement can take place in time-resolved mode. Other exemplary FRET donor/acceptor pairs include fluorescein (donor) and tetramethyl rhodamine (acceptor) with an effective distance of 55Å; IAEDANS (donor) and fluorescein (acceptor) with an effective distance of 46Å; and fluorescein (donor) and QSY-7 dye (acceptor) with an effective distance of 61Å (Molecular Probes).

Quantitative assays for nucleic acid detection also can be performed according to the present invention. The amount of secondary probe bound to a microarray spot can be measured and can be related to the amount of nucleic acid target which is in the sample. Dilutions of the sample can be used along with controls containing known amount of the target nucleic acid. The precise conditions for performing these steps will be apparent to one skilled in the art. In microarray analysis, the detectable label can be visualized or assessed by placing the probe array next to x-ray film or phosphoimagers to identify the sites where the probe has bound. Fluorescence can be detected by way of a charge-coupled device (CCD) or laser scanning.

Test Samples

Any suitable samples, including samples of human, animal, or environmental (e.g., soil or water) origin, can be analyzed using the present method. Test samples can include body fluids, such as urine, blood, semen, cerebrospinal fluid, pus, amniotic fluid, tears, or semisolid or fluid discharge (e.g., sputum, saliva, lung aspirate, vaginal or urethral discharge), stool or solid tissue samples (e.g., a biopsy or chorionic villi specimens). Test samples also include samples collected with swabs from the skin, genitalia, or throat.

Test samples can be processed to isolate nucleic acid by a variety of means well known in the art. See generally, Ausubel (Ed.) Current Protocols in Molecular Biology, 2. Preparation and Analysis of DNA and 4. Preparation and Analysis of RNA, John Wiley & Sons, Inc. (2000). It will be apparent to those skilled in the art that target nucleic acids can be RNA or DNA that may be in form of direct sample or purified nucleic acid or amplicons.

Purified nucleic acids can be extracted from the aforementioned samples and may be measured spectraphotometrically or by other instrument for the purity. For those skilled in the art of nucleic acid amplification, amplicons are obtained as end products by various amplification methods such as PCR (Polymerase Chain Reaction, U.S. Pat. Nos. 4,683,195, 4,683,202, 4,800,159 and 4,965,188), NASBA (Nucleic Acid Sequence Based Amplification, U.S. Pat. No. 5,130,238), TMA (Transcription Mediated Amplification) (Kwoh et al., Proc. Natl. Acad. Sci., USA, 86:1173-1177 (1989)), SDA (Strand Displacement Amplification, described by Walker et al., U.S. Pat. No. 5,270,184), tSDA (thermophilic Strand Displacement Amplification (U.S. Pat. No. 5,648,211 and Euro. Patent No. EP 0684315), SSSR (Self-Sustained Sequence Replication) (U.S. Pat. No. 6,156,508).

In a specific embodiment, a sample of human origin is assayed. In yet another specific embodiment, a sputum, urine, blood, tissue section, food, soil, or water sample is assayed.

Kits

The present probes can be packaged in a kit format, preferably with an instruction for using the probes to detect GBS. The components of the kit are packaged together in a common container, typically including written instructions for performing selected specific embodiments of the methods disclosed herein. Components for detection methods, as described herein, may optionally be included in the kit, for example, a second probe, and/or reagents and means for carrying out label detection (e.g., radiolabel, enzyme substrates, antibodies, etc., and the like).

The present probes, methods, and arrays can be used to detect any Streptococcus agalactiae strains that contain a cps nucleotide sequence. Exemplary S. agalactiae strains include, but are not limited to those selected from the group consisting of NCS215, NCS915, NCS535, NCS246, COH1, C388/90, CNTC 1/82, CNCTC 1/82, and NT6.

### EXAMPLES

### Example 1

### Selecting Probes for the Detection of Streptococcus agalactiae

Probes were designed that would hybridize with the GBS capsular polysaccharide synthesis (cps) gene with high specificity and sensitivity in a way to avoid false positive results. Various sub-regions of the cps gene that appeared to be conserved among the GBS were identified as potential probe sites, and are given by SEQ ID NOs:2 to 8. All nucleic acid databases in GenBank were search using the BLASTN 2.1.3 program, available online at the NCBI web site. The databases were searched using the GenBank Accession Nos. AFB028896 and AF355776 as a query sequence with either multiple or pair wise alignments, and with Expect Value at 10 (relatively more permissive) initially.

Searches were carried out at Expect Value 1 (relatively less permissive). The searches produced seven regions with significant Blast Hits to explore for designing the probes. These were the regions between nucleotides 125-200 (SEQ ID NO:2) (GenBank AFB028896); nucleotides 500-700 (SEQ ID NO:3) (GenBank AFB028896); nucleotides 10,100-10,440 (SEQ ID NO:4) (GenBank AFB028896); nucleotides 14,900-15,150 (SEQ ID NO:5) (GenBank AFB028896); nucleotides 15,300-15,475 (SEQ ID NO:6) (GenBank AF355776); nucleotides 15,600-15,700 (SEQ ID NO:7) (GenBank AF355776); and nucleotides 15,850-16,000 (SEQ ID NO:8) (GenBank AF355776). Several scans of each of these regions were analyzed and the consensus regions of the Blast Hits for the GBS were studied.

After an intensive analysis for hybridization characteristics, secondary hairpin structure and thermal profiles, several probes were selected from different sub-regions that were highly specific for cps genes. The sequences of these potential probes were then used as query sequences and compared to all nucleic acid databases in GenBank. The probes having significant identity with sequences of other genes in different organisms were eliminated and six probes were analyzed for their base composition, G+C content, thermal stability, hybridization characteristics and absence of hairpin structure formation.

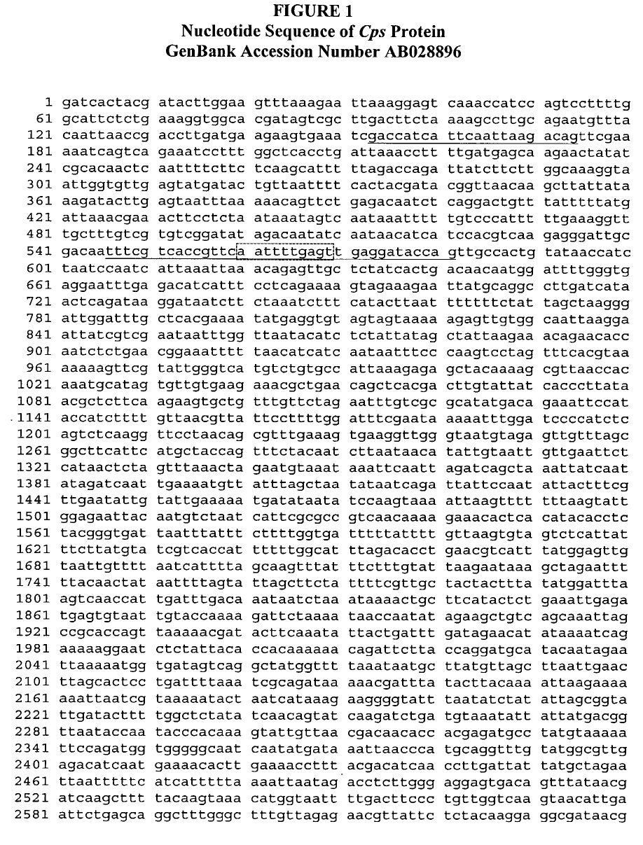

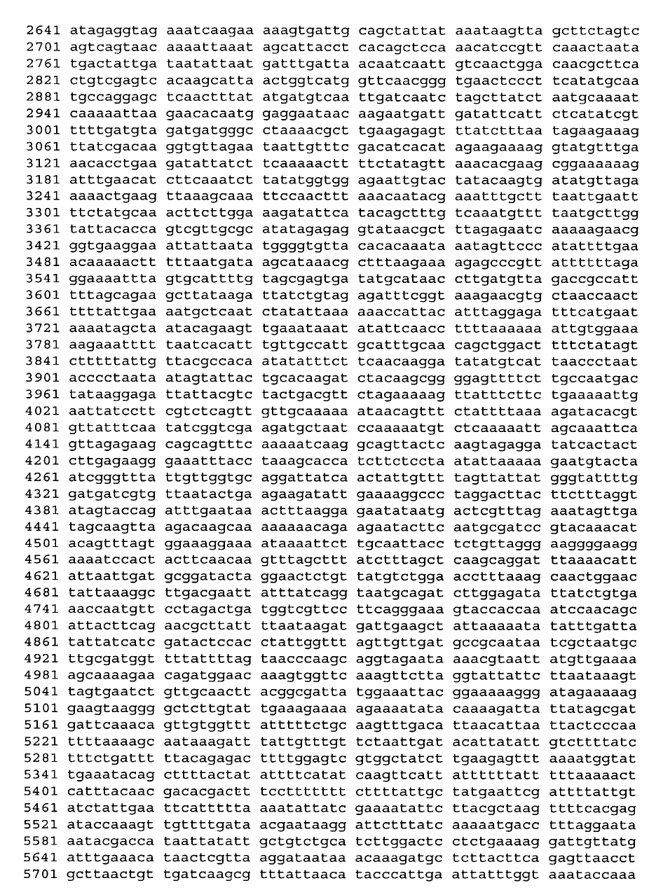

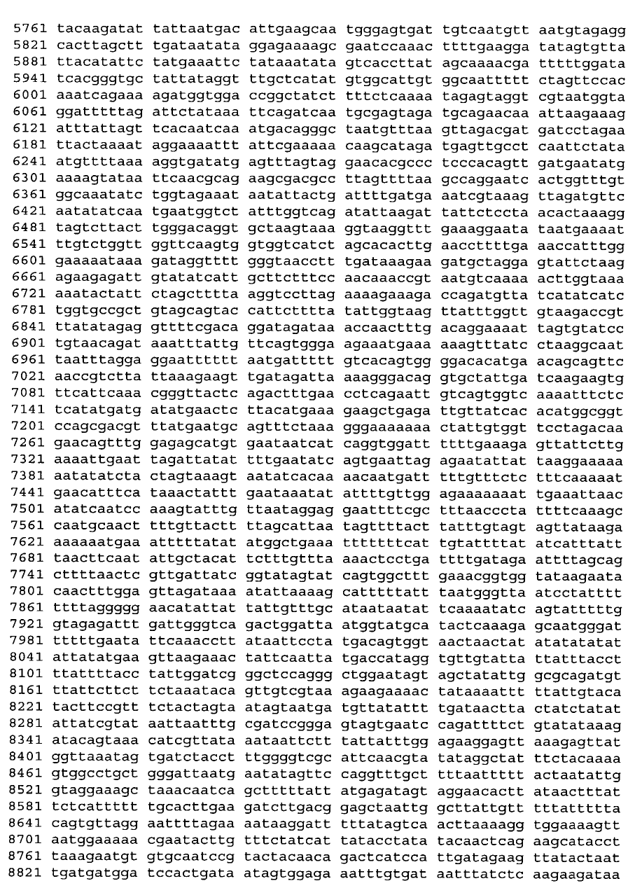

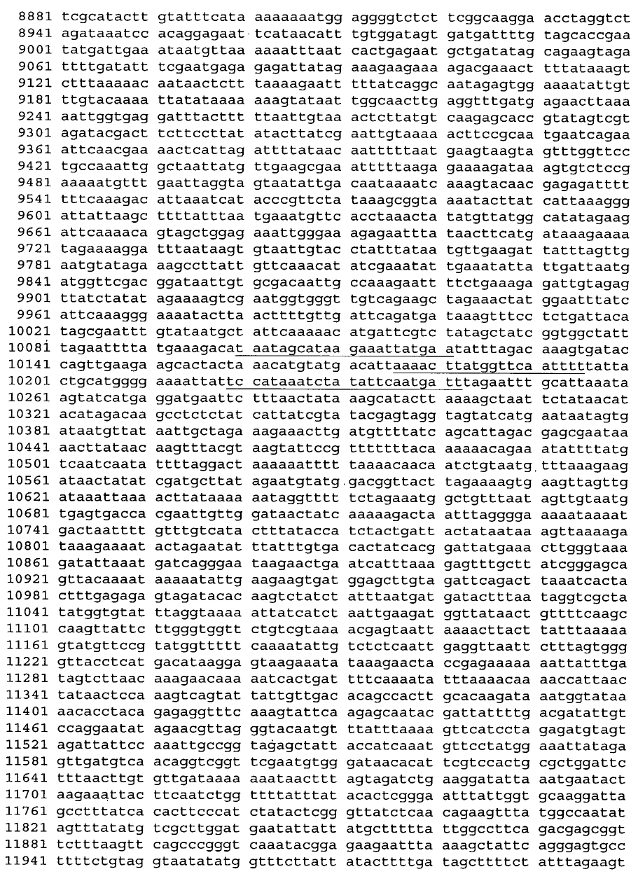

Three of these probes (AGT03007, AGT03008 and AGT03009) (SEQ ID NOs: 9, 10 and 11, respectively) were synthesized. Referring to FIG. 1, AGT03007 is an oligonucleotide sequence starting at nucleotide # 559 and ending at nucleotide # 581, AGT03008 is an oligonucleotide sequence starting at nucleotide #546 and ending at nucleotide # 569 and AGT03009 is an oligonucleotide sequence starting at nucleotide # 10,218 and ending at nucleotide # 10,242. They are as follows:

(SEQ ID NO:9; a.k.a. AGT03007)

5′-GCCGCACCAGTTAAAAACGATAC-3′,

(SEQ ID NO:10; a.k.a. AGT03008)

5′-TGTCAGCAAATTAGCCGCACCAGT-3′,

(SEQ ID NO:11; a.k.a. AGT03009)

5′-GAGTTGATGTCAACAGGTCGGTTCG-3′,

Two additional probes (AGT03010 and AGT03011) were also synthesized. Referring to FIG. 2, AGT03010 is an oligonucleotide sequence starting at nucleotide # 15,339 and ending at nucleotide # 15,360 and AGT03011 is an oligonucleotide sequence starting at nucleotide # 15,972 and ending at nucleotide # 15,996. They are as follows:

(SEQ ID NO:17; a.k.a. AGT03010)

5′-AGTCATGCTGATGCGATTGCTC-3′,

(SEQ ID NO:18; a.k.a. AGT03011)

5′-GCGATCAGGGTCAAAAGGATTACCA-3′,

### Example 2

### Demonstration of Specificity of Probes

The following example is to demonstrate specificity of the probes AGT03007, AGT03008, AGT03009, AGT03010 and AGT0301 1. The experiments were carried out by labeling genomic nucleic acid samples with a photo-chemically activatable compound. The labeled nucleic acid samples were then hybridized with the probes which were chemically immobilized to magnetic particles. The hybridized materials were detected by chemiluminescence of the label.

More particularly, clinical isolates were obtained from ATCC and cultured in the laboratory. Nucleic acids were isolated from the clinical isolates by lysing the cells with Triton X-100 in a tris-EDTA buffer. Further purification of the nucleic acid was carried out by phenol-chloroform extraction and ethanol precipitation. The precipitated DNA was dissolved in water 1 mg/ml).

The labeling compound was synthesized by the methods described in Dattagupta et al., U.S. Pat. No. 6,242,188 B1. The compound APA was synthesized by following the procedure described in Example 17 of U.S. Pat. No. 6,242,188 except in step 5 of the synthesis, methyl flurosulfonate succinimdo acridine (described in Example 19, step 3) was used instead of biotin compound. The compound was dissolved in ethanol (10 mg/ml).

Ten μl of APA was added to 100 μl of DNA solution (1 mg/ml). The solution was irradiated for 20 minutes using a hand held long wavelength (365 nm) UV lamp at room temperature. After the labeling reaction, excess unbound APA was removed by ethanol precipitation. The labeled sample was then hybridized with immobilized probes at 83° C. for 10 minutes in hybridization buffer (100 mM NaCl, 3% triton X-102, 50 mM PIPES, pH 6.5) and washed 4 times using the wash buffer (20 mM NaCl, 3% triton X-102, 50 mM PIPES, pH 6.5) at the hybridization temperature. The hybrid was detected by chemiluminescence from acridinium ester label in a commercial luminometer (Zylux, Maryville, Tenn.).

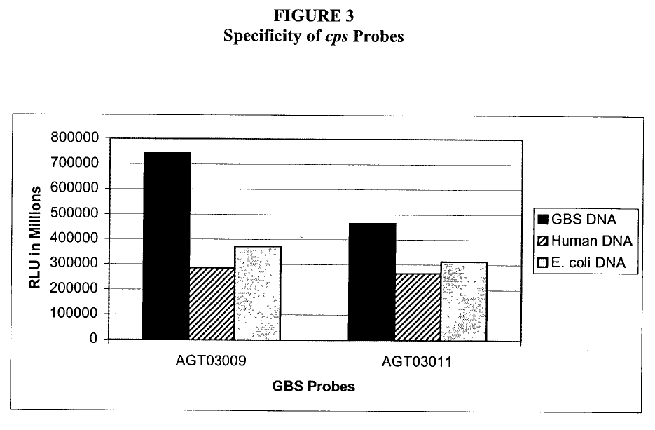

The ability of the oligonucleotide probes to detect the presence of GBS was tested using APA-labeled DNA from GBS cultures as a positive control. The APA-labeled human and E. coli DNAs were used as negative controls. The results shown in FIG. 3 demonstrate that AGT03009 and AGT03011 probes were specific for GBS.

### Example 3

### PCR and Hybridization of Probes

PCR can be conducted with any known software, e.g., Primer III (www.genome.wi.mit.edu). Two sets of primers for PCR are designed from the cps gene sequence. Sequences of the first set of primers are 5′-TTGTTGACACAGCCACTTGCAC-3′ (SEQ ID NO:36) and 5′-TGCAACAGATGAAACTGCATTGG-3′ (SEQ ID NO:37), respectively. Sequences of the second set of primers are 5′-GCCAATTTGTATTATTCCTGCG-3′ (SEQ ID NO:38) and 5′-ACAAAATTCTGTTAGCCATTGATTG-3′ (SEQ ID NO:39), respectively. These primers are checked for self-dimerization and hairpin formation abilities using an online program such as Oligo Analyzer version 2.5 (www.idtdna.com).

Two separate amplification reactions are set up with 10 μl of S. agalactiae target DNA (10 pg/μl), 1.5 μl of each primer (10 pmol/μl), 2 μl of 2.5 mM dNTP, 2.5 μl of 10× reaction buffer, and 0.2 μl of Taq DNA polymerase (5U/μl) in a 25 μl of final mixture. PCR is carried out in a thermal cycler with the following conditions: 94° C. for 5 min followed by 28 cycles of [94° C. for 1 min, 65° C. for 1 min, 72° C. for 1 min] followed by 72° C. for 10 min. The reaction mixture reveals the presence of a ˜800 bp amplicon with the first set of primers and ˜1,100 bp amplicon with the second set of primers (PCR product) by agarose gel electrophoresis. The PCR product is purified using QIAquick PCR purification kit (QIAGEN, CA, Cat. #28104). Probes AGT03009 and AGT03011 are commercially biotinylated (GenBase, CA). Under the hybridization conditions described in the Example 2, the biotynlated-oligonucleotide probes hybridize with the immobilized purified PCR products indicating specificity for the cps gene.

The examples set forth above are provided to give those of ordinary skill in the art with a complete disclosure and description of how to make and use the preferred embodiments of the compositions, and are not intended to limit the scope of what the inventors regard as their invention. Modifications of the above-described modes for carrying out the invention that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. An oligonucleotide probe for detecting Streptococcus agalactiae in a sample comprising a nucleotide sequence having at least 90% identity with a sequence selected from the group consisting of:

| | |
|---|---|
| 5′-GCCGCACCAGTTAAAAACGATAC-3′, | (SEQ ID NO:9) |
| 5′-TGTCAGCAAATTAGCCGCACCAGT-3′, | (SEQ ID NO:10) |
| 5′-GAGTTGATGTCAACAGGTCGGTTCG-3′, | (SEQ ID NO:11) |
| 5′-GTCTAATCATTCGCGCCGTCAA-3′, | (SEQ ID NO:12) |
| 5′-CCAGGAATATAGAACGTTAGGG-3′, | (SEQ ID NO:13) |
| 5′-GCCGGTAGAGCTATTACCATC-3′, | (SEQ ID NO:14) |
| 5′-CAGCATTGTCTGATAAAGGCTATGC-3′, | (SEQ ID NO:15) |
| 5′-GATATGGTGGGAGCAGATAAAGG-3′, | (SEQ ID NO:16) |
| 5′-AGTCATGCTGATGCGATTGCTC-3′, | (SEQ ID NO:17) |
| 5′-GCGATCAGGGTCAAAAGGATTACCA-3′, | (SEQ ID NO:18) |
| 5′-CGGAGCATGATGGTTATCCTG-3′, | (SEQ ID NO:19) |
| 5′-CACCAGATAGTATTTGTGGACGTGG-3′, | (SEQ ID NO:20) |
| 5′-ACCTATGTAGGGGTTCCAGCAA-3′, | (SEQ ID NO:21) |
| 3′-CGGCGTGGTCAATTTTTGCTATC-5′, | (SEQ ID NO:22) |
| 3′-ACAGTCGTTTAATCGGCGTGGTCA-5′, | (SEQ ID NO:23) |
| 3′-CTCAACTACAGTTGTCCAGCCAAGC-5′, | (SEQ ID NO:24) |
| 3′-CAGATTAGTAAGCGCGGCAGTT-5′, | (SEQ ID NO:25) |
| 3′-GGTCCTTATATCTTGCAATCCC-5′, | (SEQ ID NO:26) |
| 3′-CGGCCATCTCGATAATGGTAG-3′, | (SEQ ID NO:27) |
| 3′-GTCGTAACAGACTATTTCCGATACG-3′, | (SEQ ID NO:28) |
| 3′-CTATACCACCCTCGTCTATTTCC-5′, | (SEQ ID NO:29) |
| 3′-TCAGTACGACTACGCTAACGAG-5′, | (SEQ ID NO:30) |
| 3′-CGCTAGTCCCAGTTTTCCTAATGGT-5′, | (SEQ ID NO:31) |
| 3′-GCCTCGTACTACCAATAGGAC-3′, | (SEQ ID NO:32) |
| 3′-GTGGTCTATCATAAACACCTGCACC-3′, and | (SEQ ID NO:33) |
| 3′-TGGATACATCCCCAAGGTCGTT-3′. | (SEQ ID NO:34) |

2. The oligonucleotide probe of claim 1, further comprising a nucleotide sequence having 100% identity with a sequence selected from the group consisting of:

| | |
|---|---|
| 5′-GCCGCACCAGTTAAAAACGATAC-3′, | (SEQ ID NO:9) |
| 5′-TGTCAGCAAATTAGCCGCACCAGT-3′, | (SEQ ID NO:10) |
| 5′-GAGTTGATGTCAACAGGTCGGTTCG-3′, | (SEQ ID NO:11) |
| 5′-GTCTAATCATTCGCGCCGTCAA-3′, | (SEQ ID NO:12) |
| 5′-CCAGGAATATAGAACGTTAGGG-3′, | (SEQ ID NO:13) |
| 5′-GCCGGTAGAGCTATTACCATC-3′, | (SEQ ID NO:14) |
| 5′-CAGCATTGTCTGATAAAGGCTATGC-3′, | (SEQ ID NO:15) |
| 5′-GATATGGTGGGAGCAGATAAAGG-3′, | (SEQ ID NO:16) |
| 5′-AGTCATGCTGATGCGATTGCTC-3′, | (SEQ ID NO:17) |
| 5′-GCGATCAGGGTCAAAAGGATTACCA-3′, | (SEQ ID NO:18) |
| 5′-CGGAGCATGATGGTTATCCTG-3′, | (SEQ ID NO:19) |
| 5′-CACCAGATAGTATTTGTGGACGTGG-3′, | (SEQ ID NO:20) |
| 5′-ACCTATGTAGGGGTTCCAGCAA-3′, | (SEQ ID NO:21) |
| 3′-CGGCGTGGTCAATTTTTGCTATC-5′, | (SEQ ID NO:22) |
| 3′-ACAGTCGTTTAATCGGCGTGGTCA-5′, | (SEQ ID NO:23) |
| 3′-CTCAACTACAGTTGTCCAGCCAAGC-5′, | (SEQ ID NO:24) |
| 3′-CAGATTAGTAAGCGCGGCAGTT-5′, | (SEQ ID NO:25) |
| 3′-GGTCCTTATATCTTGCAATCCC-5′, | (SEQ ID NO:26) |
| 3′-CGGCCATCTCGATAATGGTAG-3′, | (SEQ ID NO:27) |
| 3′-GTCGTAACAGACTATTTCCGATACG-3′, | (SEQ ID NO:28) |
| 3′-CTATACCACCCTCGTCTATTTCC-5′, | (SEQ ID NO:29) |
| 3′-TCAGTACGACTACGCTAACGAG-5′, | (SEQ ID NO:30) |
| 3′-CGCTAGTCCCAGTTTTCCTAATGGT-5′, | (SEQ ID NO:31) |
| 3′-GCCTCGTACTACCAATAGGAC-3′, | (SEQ ID NO:32) |
| 3′-GTGGTCTATCATAAACACCTGCACC-3′, and | (SEQ ID NO:33) |
| 3′-TGGATACATCCCCAAGGTCGTT-3′. | (SEQ ID NO:34) |

3. The probe of claim 1, which comprises DNA, RNA, PNA or a derivative thereof.

4. The probe of claim 1, which comprises both DNA and RNA or derivatives thereof.

5. The probe of claim 1, which is labeled.

6. The probe of claim 5, wherein the label is selected from the group consisting of a chemical, an enzymatic, an immunogenic, a radioactive, a fluorescent, a luminescent and a FRET label.

7. An array of oligonucleotide probes immobilized on a support for detecting Streptococcus agalactiae, which array comprises a support suitable for use in nucleic acid hybridization having immobilized thereon a plurality of oligonucleotide probes, at least one of said probes being a probe according to claim 1.

8. The array of claim 7, wherein the plurality of probes comprise DNA, RNA, PNA or a derivative thereof.

9. The array of claim 7, wherein at least one of the probes comprises both DNA and RNA or derivatives thereof.

10. An array of oligonucleotide probes immobilized on a support for detecting Streptococcus agalactiae, which array comprises a support suitable for use in nucleic acid hybridization having immobilized thereon a plurality of oligonucleotide probes, at least one of said probes being a probe according to claim 2.

11. The array of claim 7, wherein at least one of the probes is labeled.

12. The array of claim 11, wherein the label is selected from the group consisting of a chemical, an enzymatic, an immunogenic, a radioactive, a fluorescent, a luminescent and a FRET label.

13. The array of claim 7, wherein the support comprises a surface that is selected from the group consisting of a silicon, a plastic, a glass, a ceramic, a rubber, and a polymer surface.

14. A method for detecting Streptococcus agalactiae in a sample, comprising the steps of:
a) providing an oligonucleotide probe comprising a nucleotide sequence, or a complementary strand thereof, having at least 90% identity with a sequence selected from the group consisting of:

| | |
|---|---|
| 5′-GCCGCACCAGTTAAAAACGATAC-3′, | (SEQ ID NO:9) |
| 5′-TGTCAGCAAATTAGCCGCACCAGT-3′, | (SEQ ID NO:10) |
| 5′-GAGTTGATGTCAACAGGTCGGTTCG-3′, | (SEQ ID NO:11) |
| 5′-GTCTAATCATTCGCGCCGTCAA-3′, | (SEQ ID NO:12) |
| 5′-CCAGGAATATAGAACGTTAGGG-3′, | (SEQ ID NO:13) |
| 5′-GCCGGTAGAGCTATTACCATC-3′, | (SEQ ID NO:14) |
| 5′-CAGCATTGTCTGATAAAGGCTATGC-3′, | (SEQ ID NO:15) |
| 5′-GATATGGTGGGAGCAGATAAAGG-3′, | (SEQ ID NO:16) |
| 5′-AGTCATGCTGATGCGATTGCTC-3′, | (SEQ ID NO:17) |
| 5′-GCGATCAGGGTCAAAAGGATTACCA-3′, | (SEQ ID NO:18) |
| 5′-CGGAGCATGATGGTTATCCTG-3′, | (SEQ ID NO:19) |
| 5′-CACCAGATAGTATTTGTGGACGTGG-3′, | (SEQ ID NO:20) |
| 5′-ACCTATGTAGGGGTTCCAGCAA-3′, | (SEQ ID NO:21) |
| 3′-CGGCGTGGTCAATTTTTGCTATC-5′, | (SEQ ID NO:22) |
| 3′-ACAGTCGTTTAATCGGCGTGGTCA-5′, | (SEQ ID NO:23) |
| 3′-CTCAACTACAGTTGTCCAGCCAAGC-5′, | (SEQ ID NO:24) |
| 3′-CAGATTAGTAAGCGCGGCAGTT-5′, | (SEQ ID NO:25) |
| 3′-GGTCCTTATATCTTGCAATCCC-5′, | (SEQ ID NO:26) |
| 3′-CGGCCATCTCGATAATGGTAG-3′, | (SEQ ID NO:27) |
| 3′-GTCGTAACAGACTATTTCCGATACG-3′, | (SEQ ID NO:28) |
| 3′-CTATACCACCCTCGTCTATTTCC-5′, | (SEQ ID NO:29) |
| 3′-TCAGTACGACTACGCTAACGAG-5′, | (SEQ ID NO:30) |
| 3′-CGCTAGTCCCAGTTTTCCTAATGGT-5′, | (SEQ ID NO:31) |
| 3′-GCCTCGTACTACCAATAGGAC-3′, | (SEQ ID NO:32) |
| 3′-GTGGTCTATCATAAACACCTGCACC-3′, and | (SEQ ID NO:33) |
| 3′-TGGATACATCCCCAAGGTCGTT-3′; | (SEQ ID NO:34) |

b) contacting said probe with a sample containing or suspected of containing a Streptococcus agalactiae target nucleotide sequence under conditions suitable for hybridization between said probe and said target nucleotide sequence; and
c) assessing hybridization between said probe and said target nucleotide sequence to detect said Streptococcus agalactiae in said sample.

15. The method of claim 14, wherein the probe comprises DNA, RNA, PNA or a derivative thereof.

16. The method of claim 14, wherein the probe comprises both DNA and RNA or derivatives thereof.

17. The method of claim 14, wherein the probe comprises a nucleotide sequence that is selected from the group consisting of:

| | |
|---|---|
| 5′-GCCGCACCAGTTAAAAACGATAC-3′, | (SEQ ID NO:9) |
| 5′-TGTCAGCAAATTAGCCGCACCAGT-3′, | (SEQ ID NO:10) |
| 5′-GAGTTGATGTCAACAGGTCGGTTCG-3′, | (SEQ ID NO:11) |
| 5′-GTCTAATCATTCGCGCCGTCAA-3′, | (SEQ ID NO:12) |
| 5′-CCAGGAATATAGAACGTTAGGG-3′, | (SEQ ID NO:13) |
| 5′-GCCGGTAGAGCTATTACCATC-3′, | (SEQ ID NO:14) |
| 5′-CAGCATTGTCTGATAAAGGCTATGC-3′, | (SEQ ID NO:15) |
| 5′-GATATGGTGGGAGCAGATAAAGG-3′, | (SEQ ID NO:16) |
| 5′-AGTCATGCTGATGCGATTGCTC-3′, | (SEQ ID NO:17) |
| 5′-GCGATCAGGGTCAAAAGGATTACCA-3′, | (SEQ ID NO:18) |
| 5′-CGGAGCATGATGGTTATCCTG-3′, | (SEQ ID NO:19) |
| 5′-CACCAGATAGTATTTGTGGACGTGG-3′, | (SEQ ID NO:20) |
| 5′-ACCTATGTAGGGGTTCCAGCAA-3′, | (SEQ ID NO:21) |
| 3′-CGGCGTGGTCAATTTTTGCTATC-5′, | (SEQ ID NO:22) |
| 3′-ACAGTCGTTTAATCGGCGTGGTCA-5′, | (SEQ ID NO:23) |
| 3′-CTCAACTACAGTTGTCCAGCCAAGC-5′, | (SEQ ID NO:24) |
| 3′-CAGATTAGTAAGCGCGGCAGTT-5′, | (SEQ ID NO:25) |
| 3′-GGTCCTTATATCTTGCAATCCC-5′, | (SEQ ID NO:26) |
| 3′-CGGCCATCTCGATAATGGTAG-3′, | (SEQ ID NO:27) |
| 3′-GTCGTAACAGACTATTTCCGATACG-3′, | (SEQ ID NO:28) |
| 3′-CTATACCACCCTCGTCTATTTCC-5′, | (SEQ ID NO:29) |
| 3′-TCAGTACGACTACGCTAACGAG-5′, | (SEQ ID NO:30) |
| 3′-CGCTAGTCCCAGTTTTCCTAATGGT-5′, | (SEQ ID NO:31) |
| 3′-GCCTCGTACTACCAATAGGAC-3′, | (SEQ ID NO:32) |
| 3′-GTGGTCTATCATAAACACCTGCACC-3′, and | (SEQ ID NO:33) |
| 3′-TGGATACATCCCCAAGGTCGTT-3′. | (SEQ ID NO:34) |

18. The method of claim 14, wherein the probe or the Streptococcus agalactiae target nucleotide sequence is labeled.

19. The method of claim 18, wherein the label is selected from the group consisting of a chemical, an enzymatic, an immunogenic, a radioactive, a fluorescent, a luminescent and a FRET label.

20. The method of claim 14, wherein the probe or the Streptococcus agalactiae target nucleotide sequence is immobilized on a support.

21. The method of claim 14, wherein a plurality of the probes immobilized on a support is used.

22. The method of claim 14, wherein a plurality of samples is assayed.

23. The method of claim 22, wherein the plurality of samples is assayed simultaneously.

24. The method of claim 14, wherein a sample of human origin is assayed.

25. The method of claim 14, wherein the sample is selected from the group consisting of sputum, urine, blood, tissue section, food, soil and water sample.

26. The method of claim 14, wherein the Streptococcus agalactiae strain is selected from the group consisting of: NCS215, NCS915, NCS535, NCS246, COH1, C388/90, CNTC 1/82, CNCTC 1/82, and NT6.

27. A method for detecting Streptococcus agalactia in a sample comprising the steps of:
a) providing an oligonucleotide probe comprising a nucleotide sequence that hybridizes with a target nucleotide sequence, wherein the target nucleotide sequence is selected from the group consisting of:
(SEQ ID NO:2)
5′-AACCGACCTTGATGAAGAAGTGAAATCGACCATCATTCAATTAAGAC
AGTTCGAAAAATCAGTCA GAAATCCTTT-3′;
(SEQ ID NO:3)
5′-TAGACAATATCAATAACATCATCCACGTCAAGAGGGATTGCGACAAT
TTCGTCACCGTTCAATTTTGAGTTGAGGATACCAGTTGCCACTGTATAAC
CATCTAATCCAATCATTAAATTAAACAGAGTTGCTCTATCACTGACAACA
ATGGATTTTGGGTGAGGAATTTGAGACATCATTTCCTCAGAAAAGTAGAA
AGAA-3′;
(SEQ ID NO:4)
5′-TAATAGCATAAGAAATTATGAATATTTAGACAAAGTGATACCAGTTG
AAGAAGCACTACTAAACATGTATGACATTAAAACTTATGGTTCAATTTTT
ATTACTGCATGGGGAAAATTATTCCATAAATCTATATTCAATGATTTAGA
ATTTGCATTAAATAAGTATCATGAGGATGAATTCTTTAACTATAAAGCAT
ACTTAAAAGCTAATTCTATAACATACATAGACAAGCCTCTCTATCATTAT
CGTATACGAGTAGGTAGTATCATGAATAATAGTGATAATGTTATAATTGC
TAGAAAGAAACTTG ATGTTTTATCAGCATTAGACGAGCGAATAA-3′;
(SEQ ID NO:5)
5′-TCCAGTGTCTATTTATAGGATCCAATTCAGATACTGGTTCTGATGAT
ATTGCAAAGGCTATCAATACTTATTTGATAAATCATGAAAATAGTTATTG
CTTTGCTTCTTTATCAACCCAACTTTATCATTCCTTAATTCGTCATTCAC
TAGGACTGATTGGTAATTCGTCTTCTGGTTTAATAGAAGTTCCTTCTTTG
ATGAAACCTACTCTTAATATTGGTGATCGCCAAAAAGGTAGGCTACATGG
AGAA-3′;
(SEQ ID NO:6)
5′-TCTAAAATGAAAAAAAAGTTGGCATTTTTAGGCGCAGGGAGTCATGC
TGATGCGATTGCTCCGGTTATTGATCCTCTTGTGTATGATTTCGTAGGTT
TTTTTGATGATAAAGATATTACGGAGCATGATGGTTATCCTGTTCTTGGA
AAACTTTATGATGTGCTACCTTACCTTGA-3′;
(SEQ ID NO:7)
5′-GTATTGACACCAGATAGTATTTGTGGACGTGGTATCTTTATTGGTTT
TGGGGCTTTTATAGGTTCTAAAGTGAAGCTGTTTGATAACAACGTTGTTA
ATAC-3′; and
(SEQ ID NO:8)
5′-CGACAATTGGAGCAGGAGCTGTTGTTGTAAAAGATATTATAGAACCT
GGAACCTATGTAGGGGTTCCAGCAAAAAAAATAAAATAAAGGATATGTTA
TGAAGCCAATTTGTATTATTCCTGCGCGATCAGGGTCAAAAGGATTACCA
GATA-3′;
or a complementary strand thereof, and wherein the probe has a G+C content from about 30 to 70%, a Tm value from about 40 to 80° C., a length of at least 8 nucleotides and does not contain any hairpin secondary structure;
b) contacting said probe with a sample containing or suspected of containing a Streptococcus agalactia target nucleotide sequence under conditions suitable for hybridization between said probe and said target nucleotide sequence; and
c) assessing hybridization between said probe and said target nucleotide sequence to detect said Streptococcus agalactia in said sample.

28. An oligonucleotide probe for detecting Streptococcus agalactiae in a sample comprising a nucleotide sequence that hybridizes with a target nucleotide sequence selected from the group consisting of:
(SEQ ID NO:2)
5′-AACCGACCTTGATGAAGAAGTGAAATCGACCATCATTCAATTAAGAC
AGTTCGAAAAATCAGTCA GAAATCCTTT-3′;
(SEQ ID NO:3)
5′-TAGACAATATCAATAACATCATCCACGTCAAGAGGGATTGCGACAAT
TTCGTCACCGTTCAATTTTGAGTTGAGGATACCAGTTGCCACTGTATAAC
CATCTAATCCAATCATTAAATTAAACAGAGTTGCTCTATCACTGACAACA
ATGGATTTTGGGTGAGGAATTTGAGACATCATTTCCTCAGAAAAGTAGAA
AGAA-3′;
(SEQ ID NO:4)
5′-TAATAGCATAAGAAATTATGAATATTTAGACAAAGTGATACCAGTTG
AAGAAGCACTACTAAACATGTATGACATTAAAACTTATGGTTCAATTTTT
ATTACTGCATGGGGAAAATTATTCCATAAATCTATATTCAATGATTTAGA
ATTTGCATTAAATAAGTATCATGAGGATGAATTCTTTAACTATAAAGCAT
ACTTAAAAGCTAATTCTATAACATACATAGACAAGCCTCTCTATCATTAT
CGTATACGAGTAGGTAGTATCATGAATAATAGTGATAATGTTATAATTGC
TAGAAAGAAACTTG ATGTTTTATCAGCATTAGACGAGCGAATAA-3′;
(SEQ ID NO:5)
5′-TCCAGTGTCTATTTATAGGATCCAATTCAGATACTGGTTCTGATGAT
ATTGCAAAGGCTATCAATACTTATTTGATAAATCATGAAAATAGTTATTG
CTTTGCTTCTTTATCAACCCAACTTTATCATTCCTTAATTCGTCATTCAC
TAGGACTGATTGGTAATTCGTCTTCTGGTTTAATAGAAGTTCCTTCTTTG
ATGAAACCTACTCTTAATATTGGTGATCGCCAAAAAGGTAGGCTACATGG
AGAA-3′;
(SEQ ID NO:6)
5′-TCTAAAATGAAAAAAAAGTTGGCATTTTTAGGCGCAGGGAGTCATGC
TGATGCGATTGCTCCGGTTATTGATCCTCTTGTGTATGATTTCGTAGGTT
TTTTTGATGATAAAGATATTACGGAGCATGATGGTTATCCTGTTCTTGGA
AAACTTTATGATGTGCTACCTTACCTTGA-3′;
(SEQ ID NO:7)
5′-GTATTGACACCAGATAGTATTTGTGGACGTGGTATCTTTATTGGTTT
TGGGGCTTTTATAGGTTCTAAAGTGAAGCTGTTTGATAACAACGTTGTTA
ATAC-3′; and
(SEQ ID NO:8)
5′-CGACAATTGGAGCAGGAGCTGTTGTTGTAAAAGATATTATAGAACCT
GGAACCTATGTAGGGGTTCCAGCAAAAAAAATAAAATAAAGGATATGTTA
TGAAGCCAATTTGTATTATTCCTGCGCGATCAGGGTCAAAAGGATTACCA
GATA-3′;
or a complementary strand thereof, and wherein the probe has a G+C content from about 30 to 70%, a Tm value from about 40 to 80° C., a length of at least 8 nucleotides and does not contain any hairpin secondary structure.

29. The probe of claim 28, which comprises DNA, RNA, PNA or a derivative thereof.

30. The probe of claim 28, which comprises both DNA and RNA or derivatives thereof.

31. The probe of claim 28, which is labeled.

32. The probe of claim 31, wherein the label is selected from the group consisting of a chemical, an enzymatic, an immunogenic, a radioactive, a fluorescent, a luminescent and a FRET label.
